(19) United States
(12) Patent Application Publication
(10) Pub. No.: US 2025/0339562 A1
(43) Pub. Date: Nov. 6, 2025

(54) Viral Vector Encoding GAD For Treating Spasticity

(71) Applicants: EG 427, Paris (FR); University of Copenhagen, Copenhagen (DK)

(72) Inventors: Grzegorz F. SAREK, Basel (CH); David J. FINK, Paris (FR); Carmelo BELLARDITA, Copenhagen (DK)

(21) Appl. No.: 19/228,717

(22) Filed: Jun. 4, 2025

Related U.S. Application Data
(63) Continuation of application No. PCT/EP2023/081871, filed on Nov. 15, 2023.

(30) Foreign Application Priority Data
Dec. 5, 2022 (FR) 2212771

(51) Int. Cl. A61K 48/00; C12N 9/88; C12N 15/86

(52) U.S. Cl. CPC A61K 48/0058; C12N 9/88; C12N 15/86; C12Y 401/01015; C12N 2710/16643

(57) ABSTRACT

The present invention provides a method for treating spasticity in a subject. The method includes direct administration of a herpes simplex virus 1 (HSV-1) vector harboring a glutamic acid decarboxylase (GAD) gene (preferably. GAD67) into one or more dermatomes of the subject.

### RELATED APPLICATIONS

This application is a continuation of International Application No. PCT/EP2023/081871, which designated the United States and was filed on Nov. 15, 2023, published in English, which claims priority to French application serial number 2212771, filed on Dec. 5, 2022, the contents of which are incorporated herein by reference in its entirety.

### SEQUENCE LISTING

The sequence listing submitted via EFS, in compliance with 37 CFR § 1.52 (e) (5), is incorporated herein by reference. The sequence listing XML file submitted via EFS contains the file “42873003US1 SubSeqListing.xml”, created on Jun. 10, 2025, which is 19,323 bytes in size.

### FIELD OF THE APPLICATION

This application relates to methods for treating spasticity.

### BACKGROUND

Spasticity is a condition in which muscles stiffen or tighten, preventing normal fluid movement. The muscles remain contracted and resist being stretched, thus affecting movement, speech and gait. Spasticity commonly occurs in disorders of the central nervous system (CNS), usually following spinal cord injury (SCI), affecting the upper motor neurons and thus is part of the upper motor neuron (UMN) syndrome. The impact of spasticity on a patient varies from a subtle neurological sign to severe spasticity (e.g., tightly clenched fists, twisted wrist and elbow joints, and fixed arms in flexed positions) causing extreme discomfort, pain, spasm, and contracture. These symptoms may be aggravated by fatigue, stress, infections, and lesions. Additionally, a patient suffering spasticity usually needs to spend extra energy to overcome muscle tone in daily activities and thus may experience increased fatigue on a daily basis.

Spasticity often requires both pharmacological and non-pharmacological interventions. The non-pharmacological interventions, such as physical therapy (i.e., stretching and strengthening exercises of muscle groups), may serve as an auxiliary treatment. On the other hand, Baclofen has been used as the prevailing pharmacological intervention for treating spasticity. Baclofen is a muscle relaxant that works on nerves in the spinal cord. Though clinical studies show Baclofen is the most potent anti-spasticity pharmacological treatment, it is often associated with side effects including drowsiness, dizziness, headache, fatigue, muscle weakness, and progressive tolerance development. Baclofen may be administrated either orally or intrathecally using a pump implanted under the skin. Since intrathecal administration requires much lower doses of Baclofen and thus reduces the side effects, it is often preferred in treating spasticity patients. However, implanted pumps may cause post-implant complications including pump failure, infection, and lead displacement. Meanwhile, the injections of Botulinum toxin (Botox) and neurolytics (phenol) have also been used, alone or combined with each other or in conjunction with the administration of Baclofen, to relieve spasticity. Botulinum toxin and neurolytic injections usually require highly trained physicians and relatively long injection times: Botulinum toxin may need to be injected into multiple muscles to show the therapeutic effect; and neurolytic needs to be injected directly on a nerve, e.g., which requires finding the nerves to be blocked for sending the messages to the muscles to contract—a subject will be sedated while a specialist uses mild electrical impulses to find the nerves. The therapeutic effects of Botulinum toxin and neurolytic injections in relieving spasticity, whether alone or in conjunction with other treatments, are short-term and require repetitive administration every 3-6 months. Neurolytic injections impair nerve conduction by destroying a portion of a nerve and often cause additional necrosis of the neighboring sensory nerves, skin, muscles, blood vessels, and other soft tissues. In addition, while the origin of spasticity affecting individual muscle groups can be somatotopically mapped to specific spinal segments, currently available intrathecal delivery may not specifically target a designated spinal segment, thus unable to reduce the side effects on other spinal segments otherwise not yet affected by spasticity.

Surgeries may be performed to section nerves and relieve spasticity in severe cases, e.g., dorsal rhyzotomy. Selective dorsal rhyzotomy consists of a spinal operation that reduces spasticity by selectively cutting sensory nerves. Sensory nerves are a main source of excitation to the spinal cord and together with the other components (spinal interneurons, motor neurons and muscles) forms close-loop neuromuscular system that naturally generates and modulates movements. However, after SCI, the affected sensory nerve will generate, amplify and reverberate neural activity inducing sustained and involuntary muscle contractions. Interruption of this close-loop system with dorsal rhizotomy has been shown to be beneficial for reducing spasticity after SCI. Nevertheless, this procedure is extremely invasive and it does not allow modulation nor preservation of sensory function in SCI patients. These surgical procedures typically reduce upper-extremity spasticity but are associated with more severe, long-term adverse effects such as sensory disturbance and decrease in motor function in the affected area.

Therefore, there exists a need for a nonsurgical, minimally invasive, region-specific, therapeutic approach to treat spasticity.

### SUMMARY OF THE INVENTION

This application provides a method of treating spasticity in a subject comprising upregulating GAD (glutamic acid decarboxylase) gene. The upregulation of GAD gene may be a region-specific upregulation of GAD gene. In some embodiments, the upregulation of GAD gene comprises administrating to the subject a viral vector comprising a polynucleotide encoding GAD, wherein GAD is expressed, converting the excitatory neurotransmitters into inhibitory neurotransmitters, thereby decreasing spasticity. In one aspect, the GAD gene is overexpressed. The polynucleotide encoding GAD may include GAD67 gene (GenBank: M81883.1; SEQ ID NO: 1) encoding a GAD67 (SEQ ID NO: 2) and GAD65 gene (GenBank: M81882.1; SEQ ID NO: 3). In a preferable embodiment, GAD is GAD67.

In some embodiments, the viral vector used in the method of this application is an adeno-associated virus (AAV) vector or a herpes simplex virus (HSV) vector, preferably an HSV-1 vector or an HSV-2 vector, more preferably a defective viral vector derived from HSV-1, such as a recombinant HSV-1 vector, an amplicon HSV-1 vector, or a HSV-1 vector comprising a pre-HSV-1 vector and a GAD expression cassette inserted. In a preferable embodiment, the viral vector used in the method of this application is a defective viral vector derived from HSV-1, wherein the polynucleotide encoding GAD is inserted in the LAT (Latency Associated Transcripts) locus in the defective viral vector derived from HSV-1.

In some embodiments, the viral vector comprises a promoter for driving the long-term expression of GAD gene. In some embodiments, promoters useful in the invention may be active selectively in afferent neurons. Such promoters can be selected from promoters of genes coding for sensory neuroreceptors, promoters of genes coding for sensory neuromodulators or sensory neurotransmitters, and promoter of genes involved in neurite outgrowth and stress response in sensory neurons. In some embodiments, promoter of genes coding for sensory neuroreceptors according to the invention is selected from promoters of the TRP gene family, more preferentially the promoter TRPV1 or TRPM8. In some embodiments, promoters of genes coding for sensory neuromodulators or sensory neurotransmitters according to the invention is selected from promoters of Substance P, PACAP, Calcitonin Gene Related Peptide (CGRP). In some embodiments, the promoter of genes involved in neurite outgrowth and stress response in sensory neurons is the promoter of the gene encoding advillin (ADVL).

In some embodiments, the promoter useful in the invention is a ubiquitous promoter, selected from human cytomegalovirus (HCMV) promoter, human elongation factor 1α (hEF-1α) promoter, β-actin promoter, rous sarcoma virus (RSV) promoter, human ubiquitin C (hUBC) promoter, ubiquitin B promoter, simian vacuolating virus 40 (SV40) promoter, phosphoglycerate kinase (PGK) promoter, β-globin promoter, NF-κB promoter, EGR1 promoter, elF4A1 promoter, FerL promoter, GAPDH promoter, β-Kin promoter, ROSA26 promoter, and human surfactant protein C (hSP-C) promoter. Preferably, the promoter used in the invention is hEF-1α (SEQ ID NO: 5).

In some embodiments, the viral vector is administered directly into the spinal parenchyma of the subject, into the intrathecal space of the subject, into the spinal subpial space of the subject, or into a peripheral spastic muscle of the subject, or into one or more dermatomes of the subject. In a preferable embodiment, the viral vector is administered directly into one or more dermatomes of the subject via one or more injections.

Also provided in this application is a method of treating spasticity in a subject comprises administrating to the subject a therapeutically effective amount of a viral vector comprising a polynucleotide encoding GAD, thereby treating spasticity in the subject. In some embodiments, the polynucleotide encoding GAD may include GAD67 gene (SEQ ID NO: 1) encoding a GAD67 (SEQ ID NO: 2) and GAD65 gene (SEQ ID NO: 3) encoding GAD65. Preferably, the GAD is GAD67.

In some embodiments, the viral vector is an adeno-associated virus (AAV) vector or a herpes simplex virus (HSV) vector, preferably an HSV-1 vector or an HSV-2 vector, more preferably a defective viral vector derived from HSV-1, such as a recombinant HSV-1 vector, an amplicon HSV-1 vector, or a HSV-1 vector comprising a pre-HSV-1 vector and a GAD expression cassette inserted. In a preferable embodiment, the viral vector used in the method of this application is a defective viral vector derived from HSV-1, wherein the polynucleotide encoding GAD is inserted in the LAT (Latency Associated Transcripts) locus in the defective viral vector derived from HSV-1.

In some embodiments, the viral vector is administered directly into the spinal parenchyma of the subject, into the intrathecal space of the subject, into the spinal subpial space of the subject, or into a peripheral spastic muscle of the subject, or into one or more dermatomes of the subject. In a preferable embodiment, the viral vector is administered directly into one or more dermatomes of the subject.

In some embodiments, the viral vector comprises a promoter for driving the long-term expression of the polynucleotide. In some embodiments, promoters useful in the invention may be active selectively in afferent neurons. Such promoters can be selected from promoters of genes coding for sensory neuroreceptors, promoters of genes coding for sensory neuromodulators or sensory neurotransmitters, and promoter of genes involved in neurite outgrowth and stress response in sensory neurons. In some embodiments, promoter of genes coding for sensory neuroreceptors according to the invention is selected from promoters of the TRP gene family, more preferentially the promoter TRPV1 or TRPM8. In some embodiments, promoters of genes coding for sensory neuromodulators or sensory neurotransmitters according to the invention is selected from promoters of Substance P, PACAP, Calcitonin Gene Related Peptide (CGRP). In some embodiments, the promoter of genes involved in neurite outgrowth and stress response in sensory neurons is the promoter of the gene encoding advillin (ADVL).

In some embodiments, the promoter useful in the invention is a ubiquitous promoter, selected from human cytomegalovirus (HCMV) promoter, human elongation factor 1α (hEF-1α) promoter, β-actin promoter, rous sarcoma virus (RSV) promoter, human ubiquitin C (hUBC) promoter, ubiquitin B promoter, simian vacuolating virus 40 (SV40) promoter, phosphoglycerate kinase (PGK) promoter, β-globin promoter, NF-κB promoter, EGR1 promoter, elF4A1 promoter, FerL promoter, GAPDH promoter, β-Kin promoter, ROSA26 promoter, and human surfactant protein C (hSP-C) promoter. Preferably, the promoter used in the invention is hEF-1α (SEQ ID NO: 5).

This application also provides a treatment regimen for treating a subject having spasticity or a condition associated with spasticity comprises administrating a viral vector comprising a polynucleotide encoding GAD, wherein GAD is expressed, thereby treating the spasticity or the condition associated with spasticity. The polynucleotide encoding GAD may include GAD67 gene (SEQ ID NO: 1) encoding a GAD67 (SEQ ID NO: 2) and GAD65 gene (SEQ ID NO: 3) encoding a GAD65. Preferably, the GAD is GAD67. The viral vector is an adeno-associated virus (AAV) vector or a herpes simplex virus (HSV) vector, preferably an HSV-1 vector or an HSV-2 vector, more preferably a defective viral vector derived from HSV, such as a recombinant HSV-1 vector, an amplicon HSV-1 vector, or a HSV-1 vector comprising a pre-HSV-1 vector and a GAD expression cassette inserted. In a preferable embodiment, the viral vector used in the method of this application is a defective viral vector derived from HSV-1, wherein the polynucleotide encoding GAD is inserted in the LAT (Latency Associated Transcripts) locus in the defective viral vector derived from HSV-1.

In some embodiments, the viral vector may be administered directly into the spinal parenchyma of the subject, into the intrathecal space of the subject, into the spinal subpial space of the subject, or into a peripheral spastic muscle of the subject, or into one or more dermatomes of the subject. Preferably, the viral vector may be administered directly into one or more dermatomes of the subject.

The viral vector may comprise a promoter. In some embodiments, promoters useful in the treatment regimen may be active selectively in afferent neurons. Such promoters can be selected from promoters of genes coding for sensory neuroreceptors, promoters of genes coding for sensory neuromodulators or sensory neurotransmitters, and promoter of genes involved in neurite outgrowth and stress response in sensory neurons. In some embodiments, promoter of genes coding for sensory neuroreceptors according to the invention is selected from promoters of the TRP gene family, more preferentially the promoter TRPV1 or TRPM8. In some embodiments, promoters of genes coding for sensory neuromodulators or sensory neurotransmitters according to the invention is selected from promoters of Substance P, PACAP, Calcitonin Gene Related Peptide (CGRP). In some embodiments, the promoter of genes involved in neurite outgrowth and stress response in sensory neurons is the promoter of the gene encoding advillin (ADVL). In some embodiments, the promoter useful in the treatment regimen is a ubiquitous promoter, selected from human cytomegalovirus (HCMV) promoter, human elongation factor 1α (hEF-1α) promoter, β-actin promoter, rous sarcoma virus (RSV) promoter, human ubiquitin C (hUBC) promoter, ubiquitin B promoter, simian vacuolating virus 40 (SV40) promoter, phosphoglycerate kinase (PGK) promoter, β-globin promoter, NF-κB promoter, EGR1 promoter, elF4A1 promoter, FerL promoter, GAPDH promoter, β-Kin promoter, ROSA26 promoter, and human surfactant protein C (hSP-C) promoter.

### BRIEF DESCRIPTION OF THE DRAWINGS

FIG. 1A through FIG. 1E depicts a statistically significant reduction of the tonic muscle hyperactivity when mice are treated with hEF-1α::GAD67 vector. Representative images of mouse tails in lesioned control animals (FIG. 1A) and treated animals (FIG. 1B) in the chronic state of SCI, 3 weeks after the injection of the vectors. FIG. 1C shows a severity index characterizing the increase of the tonic stretch exaggeration leading to abnormal posture quantification. FIG. 1D shows severity index of each group (N=10 animals per group). Each dot represents one animal and bar graphs represent mean±standard deviation. Statistical analysis with unpaired t-Test shows a significant difference (p<0.05) between the treated mice compared to the lesioned control ones, where the severity index is significantly decreased after hEF1α-hGAD67 treatment. FIG. 1E shows that, when splitting the global index into its 3 contributors, only one, the first curvature of the tail θ1, shows statistically significant difference between the groups. These results tend to show a local effect on the first muscle segment (proximal to the tail base) of the hEF-1α::GAD67 vector.

FIG. 2A through FIG. 2C show mean EMG change upon tactile stimulation of the tail (tip or base) compared to baseline before stimulation. FIG. 2A shows EMG recording of the muscle at the base of the tail, allegedly related to the same metameric level as the injected dermatome (S1-S2). FIGS. 2B and 2C show the effect at 2 other tail muscles: one in the middle of the tail, and one at the tip (more caudal) of the tail.

FIG. 3A through FIG. 3C depict the EMG evaluation on spasms of the tail of a combination of the vector with a GABA re-uptake inhibitor, such as tiagabine. FIG. 3A shows the change of EMG activity of base tail muscle upon tactile stimulation at base and tip of the tail. FIGS. 3B and 3C show the effect at 2 other tail muscles: one in the middle of the tail, and one at the tip (more caudal) of the tail.

### DETAILED DESCRIPTION

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising”, which is used interchangeably with “including”, “containing”, or “characterized by”, is inclusive or open-ended language and does not exclude additional, unrecited elements or method steps.

The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally, the subject is human, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

“Drug tolerance” as used herein is meant to describe a subject's reduced reaction to a drug, usually following the repeated use of the drug. Increasing the drug's dosage may re-amplify the drug's effects; however, this may accelerate tolerance, further reducing the drug's effects. In some embodiments, the terms “tolerance”, “resistance”, and “insensitivity” may be used interchangeably to describe the reduction in effectiveness of a medication.

A “therapeutic effect,” as used herein, encompasses a therapeutic benefit and/or a prophylactic benefit as described herein.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.” Nevertheless, it will be clearly determinable, following a treatment according to the present methods.

As used herein, “treatment” or “treating” means to administer a composition to a subject or a system with an undesired condition. The condition can include a disease or disorder. “Prevention” or “preventing” means to administer a composition to a subject or a system at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “active fragment” refers to an amino acid fragment that is less than the entire amino acid sequence of the molecule and retains substantially the same biological activity or a corresponding biological activity, for example, an activity of more than 50%, such as 60%, 70%, 80%, 85%, 90%, 91%, 92%, 93%, 94%, 95%, 96%, 97%, 98%, 99% or 100%.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, a “regulatory gene” or “regulatory sequence” is a nucleic acid sequence that encodes products (e.g., transcription factors) that control the expression of other genes.

As used herein, a “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from eukaryotic mRNA, genomic DNA sequences from eukaryotic DNA, and synthetic nucleic acids. A transcription termination sequence will usually be located 3′ to the coding sequence.

The term “transgene” refers to a particular nucleic acid sequence encoding an RNA and/or a polypeptide or a portion of a polypeptide to be expressed in a cell into which the nucleic acid sequence is introduced. The term “transgene” includes (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell, or the transgene may include both a leader peptide or signal sequence plus a membrane anchor peptide, or even be a fusion protein between two naturally occurring proteins or part of them, such that the transgene will remain anchored to cell membranes, or a sequence that allows the protein to accumulate in a specific region of the cell, such as a nuclear localizing signal.

As used herein, the term “expression cassette” or “transcription cassette” refers to a distinct component of vector DNA consisting of a gene and regulatory sequence to be expressed by a transfected cell. In each successful transfection, the expression cassette directs the cell's machinery to make RNA and protein(s). Some expression cassettes are designed for modular cloning of protein-encoding sequences so that the same cassette can easily be altered to make different proteins. An expression cassette can be composed of one or more genes and the sequences controlling their expression. An expression cassette comprises at least three components: a promoter sequence, an open reading frame, and a 3′ untranslated region that, in eukaryotes, usually contains a polyadenylation site.

As used herein, a “promoter” is defined as a regulatory DNA sequence generally located upstream of a gene that mediates the initiation of transcription by directing RNA polymerase to bind to DNA and initiating RNA synthesis. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.; e.g., tissue specific promoter, cell type specific promoter, etc.), and it may be a temporally restricted promoter (i.e., the promoter is in the “ON” state or “OFF” state during specific stages of embryonic development or during specific stages of a biological process). For purposes of the present invention, a promoter sequence includes at least the minimum number of bases or elements necessary to initiate transcription of a gene of interest at levels detectable above background. Within the promoter sequence is a transcription initiation site, as well as RNA polymerase binding domains. Eukaryotic promoters will often, but not always, contain “TATA” boxes and other DNA motifs, such as “CAT” or “SP1” boxes.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M) (see, e.g., Creighton, Proteins (1984)).

A conservative substitution (also called conservative replacement or conservative mutation) may include substitution such as basic for basic, acidic for acidic, polar for polar, etc. The sets of amino acids thus derived are likely to be conserved for structural reasons. These sets can be described in the form of a Venn diagram (Livingstone C. D. and Barton G. J., “Protein sequence alignments: a strategy for the hierarchical analysis of residue conservation”, Comput. Appl. Biosci. 1993, 9, 745-756; Taylor W. R., “The classification of amino acid conservation”, J. Theor. Biol. 1986, 119, 205-218), which is incorporated herein by reference.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides that are substantially identical to the polypeptides, respectively, exemplified herein, as well as uses thereof including, but not limited to, use for treating or preventing neurological diseases or disorders, e.g., neurodegenerative diseases or disorders, and/or treating SCI. Optionally, the identity exists over a region that is at least about 50 nucleotides in length, or more preferably over a region that is 100 to 500 or 1000 or more nucleotides in length, or the entire length of the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math., 1970, 2: 482c, by the homology alignment algorithm of Needleman and Wunsch, J. Mol. Biol., 1970, 48:443, by the search for similarity method of Pearson and Lipman, Proc. Nat'l. Acad. Sci. USA, 1988, 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology, 1995, supplement).

Two examples of algorithms that are suitable for determining percent sequence identity and sequence similarity are the BLAST and BLAST 2.0 algorithms, which are described in Altschul et al., Nuc. Acids Res., 1977, 25, 3389-3402; and Altschul et al., J. Mol. Biol., 1990, 215, 403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA, 1993, 90, 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). In various embodiments, nucleic acids are isolated when purified away from other cellular components or other contaminants (e.g., other nucleic acids or proteins present in the cell) by standard techniques including, including alkaline/SDS treatment, CsCl banding, column chromatography, agarose gel electrophoresis and others well-known in the art. See e.g., F. Ausubel, et al., ed., Current Protocols in Molecular Biology, 1987, Greene Publishing and Wiley Interscience, New York. In various embodiments, a nucleic acid is, for example, DNA or RNA and may or may not contain intronic sequences. In a preferred embodiment, the nucleic acid is a cDNA molecule.

As used herein “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “neuron” includes a neuron and a portion or portions thereof (e.g., the neuron cell body, an axon, or a dendrite). The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment or methods according to the invention include cerebellar granule neurons, dorsal root ganglion neurons, and cortical neurons.

The term “neuronal degeneration” is used broadly and refers to any pathological changes in neuronal cells, including, without limitation, death or loss of neuronal cells, any changes that precede cell death, and any reduction or loss of an activity or a function of the neuronal cells. The pathological changes may be spontaneous or may be induced by any event and include, for example, pathological changes associated with apoptosis. The neurons may be any neurons, including without limitation sensory, sympathetic, parasympathetic, or enteric, e.g., dorsal root ganglia neurons, motor neurons, and central neurons, e.g., neurons from the spinal cord, including inter-neurons. Neuronal degeneration or cell loss is a characteristic of a variety of neurological diseases or disorders, e.g., neurodegenerative diseases or disorders. In some embodiments, the neuron is a sensory neuron. In some embodiments, the neuron is a motor neuron. In some embodiments, the neuron is a damaged spinal cord.

As used herein, the term “dorsal root ganglia”, also called “DRG” or “spinal ganglion” or “posterior root ganglion”, is referred to is a cluster of neurons (a ganglion) in a dorsal root of a spinal nerve. The dorsal root is the afferent sensory root and carries sensory information to the brain. The cell bodies of sensory neurons, known as first-order neurons, are located in the dorsal root ganglia. The axons of dorsal root ganglion neurons are known as afferents. In the peripheral nervous system, afferents refer to the axons that relay sensory information into the central nervous system (i.e., the brain and the spinal cord).

As used herein, the term “afferent neurons” carry information from sensory receptors of the skin and other organs to the central nervous system (i.e., brain and spinal cord), whereas “efferent neurons” carry motor information away from the central nervous system to the muscles and glands of the body. “Afferent” in this application is referred to carrying inward to a central organ or section, as nerves that conduct impulses from the periphery of the body to the brain or spinal cord.

As used herein, the term “systemic” is referred to pertaining to or affecting the whole body rather than one part of it. The term “region-specific” as used herein is referred to as only affecting a particular region (such as a particular cell type, a particular dermatome, and a particular spinal nerve) of the body and having zero or minimal impact on the rest of the body.

As used herein, the term “sensory neurons”, also known as “afferent neurons”, are neurons in the nervous system, that convert a specific type of stimulus, via their receptors, into action potentials or graded potentials. This process is called sensory transduction. The cell bodies of the sensory neurons are located in the dorsal root ganglia of the spinal cord. In some embodiments, “sensory neurons”, “sensory fibers”, and “afferent neurons” are used interchangeably in this application. Sensory neurons can be classified in various ways, such as by their morphology, location, and the stimulus they are responsible for detecting. For example, given the stimulus they are responsible for detecting, sensory neurons are classified as: olfactory sensory neurons for detecting smell, gustatory receptors for detecting taste, photoreceptors for converting light into electrical signals, thermoreceptors for detecting changes in temperature, mechanoreceptors for detecting changes in pressure or mechanical stress, proprioceptors (also called position sensors) for sensing the location of our body parts in relation to other body parts, nociceptors for processing pain and temperature sensations. As another example, according to their different sizes and degrees of myelination (and thus different conduction velocity), sensory neurons can also be classified as A, B, and C, wherein A type can be further classified into alpha, beta, gamma, and delta.

### Spasticity

As used herein, “spasticity” refers to a condition in which certain muscles exhibit hypersensitivity to stretch reflex. And “spastic muscles” can be used to describe such continuously contracted muscles due to spasticity. This contraction causes stiffness or tightness of the muscles and can interfere with normal movement, speech, and gait. Besides, symptoms of spasticity can vary from being mild stiffness or tightening of muscles to painful and uncontrollable spasms. Pain or tightness in joints is also common in spasticity. Spasticity mostly occurs in disorders of the central nervous system affecting the upper motor neurons in the form of a lesion, such as spastic diplegia, or upper motor neuron syndrome, and can also be present in various types of multiple sclerosis, where it occurs as a symptom of the progressively worsening attacks on myelin sheaths and is thus unrelated to the types of spasticity present in neuromuscular cerebral palsy rooted spasticity disorders. Without being bound by theory, spasticity develops when an imbalance occurs in the excitatory and inhibitory input to a motor neuron caused by damage to the spinal cord and/or central nervous system. The damage causes a change in the balance of signals between the nervous system and the muscles, leading to increased excitability of motor neurons triggering an overactivity in muscles. Pharmacological interventions such as Baclofen attempt to reduce the muscle excitability and thus alleviate spasticity. Spasticity is found in conditions where the brain and/or spinal cord are damaged or fail to develop normally; these include cerebral palsy, multiple sclerosis, spinal cord injury, and acquired brain injury including stroke and traumatic brain injury.

In the case of spasticity due to spinal cord injury (SCI), a maladaptive state of the spinal cord below the injury results in increase in spinal reflexes, increase in muscle tone and the appearance of involuntary tonic muscle contractions or spasms. These clinical symptoms represent the human clinical condition referred to spasticity. Independently of the type and the level of the lesion, over 70% of people with SCI experience spasticity one year after the traumatic event and it will be a lifelong, permanent motor impairment.

### Gamma-Aminobutyric Acid (GABA)

Gamma-aminobutyric acid (GABA) and glutamate are the primary inhibitory and excitatory neurotransmitters in mammals. “Neurotransmitter” is a signaling molecule secreted by a neuron to affect another cell across a synapse. Specifically, “excitatory neurotransmitters” have excitatory effects on the neuron. This means they increase the likelihood that the neuron will fire a signal called an action potential in the receiving cells. Neurotransmitters can act in predictable ways, but they can be affected by drugs, disease, and interaction with other chemical messengers. The balance between GABA and glutamate controls diverse processes such as neurogenesis, movement, circadian clocks, tissue development and blood glucose regulation. The cause of spasticity is thought to be where an imbalance occurs in the excitatory and inhibitory input to a motor neurons caused by damage to the spinal cord and/or central nervous system. Loss of GABA-mediated inhibition may play a key role in the progressive increase in spinal reflexes and the appearance of spasticity.

GABA acts at inhibitory synapses in the brain by binding to specific transmembrane receptors in the plasma membrane of both pre- and postsynaptic neuronal processes. This binding causes the opening of ion channels to allow the flow of either negatively charged chloride ions into the cell or positively charged potassium ions out of the cell. This action results in a negative change in the transmembrane potential, usually causing hyperpolarization. Two general classes of GABA receptor are known: GABAA in which the receptor is part of a ligand-gated ion channel complex, and GABAB metabotropic receptors, which are G protein-coupled receptors that open or close ion channels via intermediaries (G proteins). The mechanism of Baclofen for treating spasticity is believed to be that Baclofen acts as an agonist of GABAB receptor to modulate ion channels.

### Glutamic Acid Decarboxylase (GAD)

GABA is synthesized from glutamate by the 67 kDa and 65 kDa isoforms of an enzyme Glutamic Acid Decarboxylase (GAD67 and GAD65) with pyridoxal phosphate (PLP) as a cofactor. This process converts glutamate into GABA, i.e., it converts the principal excitatory neurotransmitter into the principal inhibitory neurotransmitter, and thus reduces neuronal excitability.

Human GAD67 and GAD65 are encoded by GAD1 gene (chromosome 2) and GAD2 gene (chromosome 10), respectively, and have been isolated and cloned by Bu et al. (1992) Proc Natl Acad Sci 89:2115-2119. Human GAD67 cDNA (GenBank: M81883.1; SEQ ID NO: 1) encodes a Mr 67,000 polypeptide, with 594 amino acid residues (Genbank Accession No. NM_000817; SEQ ID NO: 2). Human GAD65 cDNA (GenBank: M81882.1; SEQ ID NO: 3) encodes a Mr 65,000 polypeptide, with 585 amino acid residues (Genbank Accession No. NM_000818). Each of which is incorporated herein by reference.

In literature, GAD67 and GAD1 have been often used interchangeably to describe the gene that encodes GAD67, and so have been GAD65 and GAD2 for the gene that encodes GAD65. In some embodiments, GAD67 and GAD65 may be referred to the two different enzymes, Glutamic Acid Decarboxylase 67 and Glutamic Acid Decarboxylase 65, respectively. In some embodiments, GAD67 and GAD65 may be referred to the two different genes encoding the different enzymes, Glutamic Acid Decarboxylase 67 and Glutamic Acid Decarboxylase 65, respectively. In some embodiments, GAD1 and GAD2 may be referred to the two different genes encoding the different enzymes, Glutamic Acid Decarboxylase 67 and Glutamic Acid Decarboxylase 65, respectively. In some embodiments, GAD1 can be used interchangeably with GAD67; and GAD2 can be used interchangeably with GAD65.

In some embodiments, “Glutamic Acid Decarboxylase” or “GAD”, as used herein, may comprise a wild type or modified GAD67, a wild type or modified GAD65, and an active fragment thereof. For example, “a polynucleotide encoding GAD” may refer to a polynucleotide encoding a wild type or modified GAD67, a wild type or modified GAD65, or an active fragment thereof.

This application provides a method of treating spasticity in a subject comprising upregulating GAD gene, thereby treating spasticity in the subject. In some embodiments, GAD may include a wild type or modified GAD67, a wild type or modified GAD65, or an active fragment thereof. In some embodiments, GAD is a wild type or modified GAD67 or an active fragment thereof.

### Herpes Simplex Virus (HSV)

A gene therapy capable of upregulating GAD genes (including GAD1/GAD67 and GAD2/GAD65) may provide a therapeutic approach for treating spasticity, wherein a viral vector is used for the delivery of a therapeutic gene product, such as a wild type or modified GAD or an active segment thereof.

The term “viral vector” or “viral expression vector” as used herein refers to a nucleic acid vector that includes at least one element of a virus genome and may be packaged into a viral particle. In the context of the present invention, the term “viral vector” has to be understood broadly as including nucleic acid vector (e.g., DNA viral vector) as well as viral particles generated thereof. In this application, the viral expression vector is an adeno-associated virus (AAV) vector or a herpes simplex virus (HSV) vector, preferably an HSV-1 vector or an HSV-2 vector, even more preferably a defective viral vector derived from HSV-1. As used herein the term “defective viral vector” shall refer to viral vectors that are missing genes or parts of genes necessary to complete successfully the viral life cycle in order to replicate.

The term “AAV” refers to the Adeno-Associated Virus itself or to derivatives thereof including recombinant AAV vector particles. Furthermore, as used herein, the term “AAV” includes many different serotypes, which have been isolated from both human and non-human primate samples. Preferred AAV serotypes are the human serotypes, more preferably human AAV of serotypes 2, 5 and 9, most preferably human AAV of serotype 5, which is the serotype displaying the highest level of neurotropism.

The term “herpes simplex virus (HSV)” is a complex, non-integrating DNA virus capable of infecting a very wide range of human and animal cells. HSV encompasses two serotypes, herpes simplex virus type 1 (HSV-1) and herpes simplex virus type 2 (HSV-2). The genome of HSV-1 has a size of approximately 153-kbp. It contains some 90 protein-encoding genes and more than 12 microRNA. The HSV-1 genome is composed of two unique segments, UL and US, each flanked by inverted repeats that encode critical diploid genes.

The term “defective viral vector derived from HSV” comprises defective recombinant HSV vectors, amplicon HSV vectors, and “pre-HSV-1 vector” as defined herein. The terms “defective recombinant HSV”, as used herein, describes a helper-independent vector, the genome of which comprises at least complete deletions of the genes coding for two essential proteins, known as ICP4 and ICP27. The ICP4 gene is present in two copies, located in the inverted repeated sequences known as c and c′ of the virus genome, and both copies of this gene are deleted. The gene encoding ICP27 is located in the unique long (UL) sequence of the virus genome.

Preferentially, helper-independent vectors according to the present invention carry the therapeutic transcription cassette(s) embedded into the LAT (Latency Associated Transcripts) locus which is a repeated locus that is contained in the inverted repeated sequences known as b and b′ of the virus genome. (See Berthomme et al., “Evidence for bidirectional element located downstream from the herpes virus simplex type 1 latency-associated promoter that increases its activity during latency”, JOURNAL OF VIROLOGY, 2000, 74, 3613-3622; and Berthomme et al. “Enhancer and long-term expression functions of herpes simplex virus type 1 latency-associated promoter are both located in the same region”, JOURNAL OF VIROLOGY, 2001, 75, 4386-4393; the contents of which are incorporated by reference.)

More preferentially, the transcription cassette is placed either between the Latency Associated Promoter (LAP) and the Long-Term Expression (LTE) region (site 1), or between the LTE region and the DNA insulator (INS) sequence present downstream of the LTE (site 2). Defective recombinant HSV-1 vectors according to the present invention carry transcription cassette(s) expressing the GAD genes described above in order to restore the GABA-mediated inhibition and the balance in the excitatory and inhibitory input, e.g., expressing a wild type or modified GAD67 and/or a wild type or modified GAD65 or an active fragment thereof driven by promoters. The b and b′ sequences of the virus genome are also known as TRL (Terminal Repeat L) and IRL (Internal Repeat L) respectively, while the c′ and c sequences are also known as IRS (Internal Repeat S) and TRS (Terminal Repeat S), where L and S refer respectively to the unique long (L) and unique short(S) sequences of the HSV-1 genome.

Moreover, helper-independent vectors according to the invention can comprise additional deletions in genes encoding non-essential proteins such as ICP34.5, UL55, UL56, and UL41 proteins. These defective HSV vectors are multiplied in cell lines expressing simultaneously the proteins ICP4 and ICP27 (Marconi et al, “HSV-1-derived helper-independent defective vectors, replicating vectors and amplicon vectors, for the treatment of brain diseases”, CURRENT OPINION IN DRUG DISCOVERY AND DEVELOPMENT, 13, 2010, 169-183; the contexts of which are incorporated by reference).

WO2006/050211 discloses the use of a defective HSV-1 vector for gene therapy for treating pain. However, the vectors according to the invention differ from the vector described in WO2006/050211 in several significant respects, which are important in regard to the usefulness and efficacy of the vectors according to the invention. Most important, transgenic transcription cassettes according to the invention are introduced into the LAT locus, as this region contains both the LTE and the DNA insulator sequences (INS) that confer long-term expression to the promoters driving transgene expression in transcription cassettes according to the invention, whereas the vector described in WO2006/050211 was conceived and proved for short-term action and, therefore, their transcription cassettes were not introduced into the LAT regions.

By “Amplicon or amplicon vector” it is meant a helper-dependent vector, the genome of which lacks most or all HSV genes coding for virus proteins. The genome of amplicon vectors is a concatemeric DNA composed of multiple copies in tandem of a plasmid, known as the amplicon plasmid, that carries one origin of DNA replication and one packaging signal from HSV-1 genome, in addition to transgenic DNA (i.e., transcription cassettes) of interest. Amplicon plasmids according to the present invention carry transcription cassettes expressing the GAD genes described above in order to restore the GABA-mediated inhibition and the balance in the excitatory and inhibitory input, e.g., expressing wild type or modified GAD67 and/or wild type or modified GAD65 or an active fragment thereof driven by promoters. In some embodiments, the promoters may be DRG-specific promoters as described in the present invention. In some embodiments, the promoters may be ubiquitous promoters.

In a preferred embodiment, the vector according to the invention is a defective recombinant vector lacking at least the genes coding for the essential proteins ICP4 and ICP27, preferentially a vector lacking both ICP4 and ICP27. This vector can lack other genes, coding for non-essential proteins, such as ICP34.5, UL55, UL56 and/or UL41 proteins, and carries the transcription cassette(s), embedded into the LAT regions of the vector genome.

In any embodiment as described herein, the defective recombinant vector lacks one copy of the ICP0 gene. In a preferred embodiment, the one copy of the IPC0 gene is removed among the LAT, ICP0, UL34.5 cluster from the IRL region of the HSV vector.

In some embodiments, the vector according to the invention is an amplicon vector carrying the transcription cassettes described above driven by promoters as described in other parts of this document. In a preferred embodiment, the transcription cassette according to the invention is introduced into the LAT locus. The expression “recombinant DNA” as used herein describes a nucleic acid molecule, i.e., a polynucleotide of genomic, cDNA, viral, semisynthetic, and/or synthetic origin, which, by virtue of its origin or manipulation, is not associated with all or a portion of the polynucleotide with which it is associated in nature. The term “recombinant” as used with respect to virus means a virus carrying a recombinant genome or a genome that has been manipulated to introduce mutations, deletions or one or more heterologous polynucleotides, including genes. The term “recombinant” as used with respect to a protein or polypeptide, means a polypeptide produced by expression of a recombinant nuclei acid. The term “recombinant” as used with respect to a host cell means a recombinant vector that carries recombinant DNA within the host cell or a cell that contains recombinant DNA inserted in its genome. The term “infection” refers to the ability of a viral vector to enter a host cell or organ or subject, or the ability of a gene product of the viral vector to enter a host cell.

Defective vectors derived from HSV allow to infect neighboring sensory neurons and establish latent infections in the nucleus of these neurons, located in the trigeminal or the dorsal root ganglia (DRG), depending on the site of infection. In particular, HSV-1 naturally infects sensory neurons and establishes lifelong latent infections in the nucleus of these neurons. For example, subcutaneous inoculation of HSV vectors encoding GAD67 into feet allows infecting DRG neurons which results in constitutive production of GAD and release of GABA for treating spinal cord injury pain. (See Liu et al., MOLECULAR THERAPY, 2004, Vol. 10, No. 1, 57-66.) Following injection in the dermatomes, defective vectors derived from HSV-1 as disclosed herein will reach the sensory DRG innervating the dermatomes from where they will stably express the therapeutic transgene, provided that adequate promoters drive their long-term expression. In some embodiments, the promoters may comprise both afferent neuron-specific promoters and ubiquitous promoters. In some embodiments, the promoters are ubiquitous promoters, preferably EF-1α. In some embodiments, the promoters are afferent neuron-specific promoters as disclosed in WO2017220800A1.

### Pre-HSV-1 Vector

In some embodiments, the viral vector used in the method of this application may comprise a pre-HSV-1 vector.

As used herein, a “pre-HSV-1 vector” is a mini-HSV-1 backbone (aka “mini-HSV-1” or “a pre-HSV-1 vector”), wherein non-essential genes, essential genes, or combinations thereof have been deleted to arrive at a genome comprising less than 130 kbp and greater than 75 kbp. Pre-vectors or mini vector “backbones” embodying the invention are described with the understanding that, as “backbones,” it is contemplated that a polynucleotide encoding GAD, with or without extraneous control elements, can be inserted therein. In some embodiments, GAD may be a wild type or modified GAD67, a wild type or modified GAD65, or an active fragment thereof.

As used herein, the qualifier “essential” in the expressions “essential genes” or “non-essential genes”, means that the given gene is essential (or not) for achieving multiplication and packaging of the virus genome, thus generating infectious progeny virus particles. HSV-1 essential genes include UL1, UL5-UL9, UL12, UL14, UL15, UL17-UL19, UL22, UL25-UL38, UL42, UL48, UL49, UL52-UL54, US6, ICP4 (2 copies). HSV-1 non-essential genes include ICP34.5 (2 copies), ICP0 (2 copies), LAT (2 copies), UL2-UL4, UL10, UL11, UL13, UL16, UL20, UL21, UL23, UL24, UL39, UL40, UL41, UL43-UL47, UL50, UL51, UL55, UL56, US1-US5, US7-US12.

In some embodiments, clusters of genes that could be deleted include, but are not limited to, genes UL2, UL3, UL4 (10.200-12.600); genes UL10, UL11 (23.200-25.200); gene UL16 (30.200-31.400); genes UL20, UL21 (40.800-43.700); genes UL23, UL24 (46.700-48.600); genes UL39, UL40, UL41 (86.400-92.700); genes UL43 to UL47 (94.700-103.200); genes UL50, UL51 (107.700-109.100); genes UL55, UL56 (115.400-117.100); one copy of genes LAT, ICP0, UL34.5 (IRL) (118.700-126.100); genes US2 to US5 (134.000-138.200); genes US7 to US12 (139.700-145.600); and/or the second copy of gene ICP0 when the first copy has already been removed among the LAT, ICP0, UL34.5 cluster.

In some embodiments, the mini-HSV-1 comprises a genome wherein at least 30 kbp have been deleted. In some embodiments, the mini-HSV-1 comprises a genome wherein at least 40 kbp have been deleted. In some embodiments, the mini-HSV-1 comprises a genome wherein at least 45 kbp have been deleted. In some embodiments, the mini-HSV-1 comprises a genome wherein at least 50 kbp have been deleted. In some embodiments, the mini-HSV-1 comprises a genome wherein at least 55 kbp have been deleted. In some embodiments, the mini-HSV-1 comprises a genome wherein at least 60 kbp have been deleted. In some embodiments, the mini-HSV-1 comprises a genome wherein at least 65 kbp have been deleted. In some embodiments, the mini-HSV-1 comprises a genome wherein at least 75 kbp have been deleted.

In some embodiments, the mini-HSV-1 comprises a genome wherein 25 kbp to 80 kbp have been deleted. In some embodiments, the mini-HSV-1 comprises a genome wherein 30 kbp to 75 kbp have been deleted. In some embodiments, the mini-HSV-1 comprises a genome wherein 35 kbp to 70 kbp have been deleted. In some embodiments, the mini-HSV-1 comprises a genome wherein 40 kbp to 60 kbp have been deleted.

A pre-HSV-1, wherein non-essential genes, essential genes, or combinations thereof have been deleted to arrive at a genome comprising less than 130 kbp and greater than 75 kbp, can allow a polynucleotide encoding GAD, with or without extraneous control elements, to be inserted therein. As a nonlimiting example, a recombination technique based on the markerless Red recombination system may be used to generate the scarless point mutations, deletions, and insertion of smaller and larger sequences in the recombinant viral vectors used in this invention (e.g., Tischer et al., En Passant Mutagenesis: A Two Step Markerless Red Recombination System. Chapter 30, In Vitro Mutagenesis Protocols: Third Edition, Methods in Molecular Biology, vol. 634. DOI 10.1007/978-1-60761-652-8_30, © Springer Science+Business Media, LLC 2010). In some embodiments, as used in this invention, a pre-HSV-1 vector comprises a modified HSV-1 genome wherein non-essential genes, essential genes, or combinations thereof have been deleted to arrive at a genome comprising less than 130 kbp and greater than 75 kbp.

In some embodiments, transgene of interest can be introduced into a LAT (Latency Associated Transcripts) locus, which is a repeated locus that is contained in the inverted repeated sequences known as b and b′ of the virus genome. The b and b′ sequences of the virus genome are also known as TRL (Terminal Repeat Long) and IRL (Internal Repeat Long), respectively. In some embodiments, the virus genome contains both LAT regions, one in the TRL and the other in the IRL. In some embodiments one of the LAT regions, either in the TRL or in the IRL has been deleted. In some embodiments, when the vector genome contains the two LAT loci, the transgene of interest can be introduced into both loci, in the TRL region and in the IRL region. In some embodiments, when the LAT loci in the IRL region is deleted, the transgene of interest can be introduced into the LAT locus in the TRL region only. In some embodiments, when the LAT loci in the TRL region is deleted, the transgene of interest can be introduced into the LAT locus in the IRL region only.

The LAT locus includes an upstream DNA insulator (INS) sequence, the Latency Associated Promoter (LAP), a region conferring Long-Term Expression (LTE) and a downstream DNA insulator (INS). In some embodiments, the transgene of interest is introduced either between the Latency Associated Promoter (LAP) and the Long-Term Expression (LTE) region, or between the LTE region and the DNA insulator (INS) sequence present downstream of the LTE.

Importantly, the LAT locus contains both the LTE and the DNA insulator sequences (INS) that confer long-term expression to a polynucleotide encoding GAD introduced into this site.

By “long-term expression sequence” or “long-term expression element (LTE)” it is meant a nucleotide sequence that when operably linked to a foreign DNA of interest allows for sustained expression of a gene product for more than 15 to 45 days or 30 to 45 days, or from 45 to 90 days, or from 90 to 365 days, or 365 days to several years or even during the life of the patient. Long-term expression (LTE) sequences were identified in HSV-1 as a region of the latency-associated transcripts (LAT), which originate from the LAT-associated promoter (LAP). This LTE is located downstream of the LAT transcription start site.

Indeed, viruses harboring a DNA fragment 3′ of the LAT promoter maintained detectable promoter expression throughout latency (Lokensgard et al, “The latency-associated promoter of herpes simplex virus type 1 requires a region downstream of the transcription start site for long-term expression during latency”, Journal of Virology, 1997, 71, 6714-6719; Berthomme et al., “Evidence for bidirectional element located downstream from the herpes virus simplex type 1 latency-associated promoter that increases its activity during latency”, JOURNAL OF VIROLOGY, 74, 2000, 3613-3622; and Berthomme et al. “Enhancer and long-term expression functions of herpes simplex virus type 1 latency-associated promoter are both located in the same region”, JOURNAL OF VIROLOGY, 75, 2001, 4386-4393; the contents of which are incorporated by reference.) Preferably, the LTE is comprised between about 1.5 kb to about 3 kb downstream of the LAT transcription start site (Perng et al., “The spontaneous reactivation function of the herpes simplex virus type 1 LAT gene resides completely within the first 1.5 kilobases of the 8.3-kilobase primary transcript”, JOURNAL OF VIROLOGY, 1996, 70, 976-984; the contents of which are incorporated by reference.) More recently, additional sequences, known as DNA insulators, have also been described both upstream and downstream the LTE region (Amelio et al., “A chromatin insulator-like element in the herpes simplex virus type 1 latency-associated transcription region binds CCCTC-binding factor and displays enhancer-blocking and silencing activities”, JOURNAL OF VIROLOGY, 2006, 80, 2358-2368; the contents of which are incorporated by reference.) These sequences also contribute to provide long-term expression to a given transcription cassette probably by inhibiting epigenetic silencing and are incorporated in the present invention as part of the LTE elements, to confer long-term expression to the expression cassette. Interestingly, sequences conferring long-term expression to the transcription cassette (both the LTE and the DNA insulator sequences) can be placed either upstream and/or downstream the GAD expression cassette.

Those of skill in the art will recognize that other LTE-like sequences, as well as other DNA insulator sequences, have been described and are continually being discovered. All such LTE-like sequences and DNA insulator sequences are encompassed by the present invention.

In some embodiments, one or more exogenous genes of interest is introduced into the pre-HSV-1 vector. In some embodiments, a combination of one or more HSV-1 essential genes, one or more HSV-1 non-essential genes, and one or more exogenous genes of interest is introduced into the pre-HSV-1 vector. In some embodiments, a combination of one or more HSV-1 essential genes and one or more exogenous genes of interest is introduced into the pre-HSV-1 vector. In some embodiments, a combination of one or more HSV-1 non-essential genes and one or more exogenous genes of interest is introduced into the pre-HSV-1 vector.

It is important that the pre-HSV-1 vectors used herein maintain enough of the HSV-1 genome so as to not become HSV-1 amplicons. By “Amplicon or amplicon vector” it is meant a helper-dependent vector, the genome of which lacks most or all HSV genes coding for virus proteins. The genome of amplicon vectors is a concatemeric DNA composed of multiple copies in tandem of a plasmid-known as the amplicon plasmid—that carries one origin of DNA replication and one packaging signal from HSV-1 genome. In cells expressing the full set of structural, replication and DNA packaging functions from HSV-1, resulting from the presence of an HSV-1 genome acting as helper, the amplicon plasmid is amplified by a rolling-circle mechanism into long head-to-tail concatemers that are then cleaved and packaged, up to one genome size, into HSV-1 virions (Kwong and Frenkel, 1985; Bataille and Epstein, 1997). Amplicon vectors are thus a concatemeric plasmidic DNA packaged into HSV-1 particles.

In contrast to amplicons, the pre-HSV-1 vectors of the invention are a helper-independent vector platform, which means that they do not need the presence of an HSV-1 genome acting as a helper virus for vector replication and packaging.

### HSV-1 Vector

As discussed above, the pre-vectors or mini vector “backbones” used in the method of this invention are vector templates and it is contemplated that a polynucleotide encoding GAD, with or without extraneous control elements, can be inserted therein. For example, in some embodiments, a polynucleotide encoding GAD can be introduced to the LAT region of the pre-HSV-1 vector. In embodiments according to the present invention a transcription cassette 5 expressing the GAD genes described above in order to restore the GABA-mediated inhibition and the balance in the excitatory and inhibitory input, e.g., expressing wild type or modified GAD67 and/or wild type or modified GAD65 or an active fragment thereof driven by promoters can be introduced to the LAT region of the pre-HSV-1 vector.

In one embodiment, used in the method and the treatment regimen of this invention, is an HSV-1 vector comprising the pre-HSV-1 vector described herein wherein a polynucleotide encoding GAD is introduced and wherein the HSV-1 vector is capable of persistent expression of GAD. In some embodiments, the LAT region of the HSV-1 vector can be used for the introduction of a polynucleotide encoding GAD. GAD as defined above may comprise a wild type or modified GAD67, a wild type or modified GAD65, and an active fragment thereof. In a preferable embodiment, used in the method and the treatment regimen of this invention, is an HSV-1 vector comprising the pre-HSV-1 vector described herein wherein a polynucleotide encoding GAD67 (SEQ ID NO: 1) is introduced and wherein the HSV-1 vector is capable of persistent expression of the protein product GAD67 (SEQ ID NO: 2). In some embodiments, the LAT region of the HSV-1 vector can be used for the introduction of a polynucleotide encoding GAD67.

In a preferable embodiment, the HSV-1 vector includes a polynucleotide encoding GAD inserted in operable connection with one or more LTEs and/or DNA insulator sequences within the HSV vector genome. By “operably connected,” it is to be understood that the one or more LTEs and/or DNA insulator sequences permit the polynucleotide encoding GAD to be expressed in a cellular environment in which genetic elements (i.e., “genes”) otherwise present within the HSV genome are transcriptionally silent.

As described herein, the pre-HSV-1 vector of the invention comprises deletions to the HSV genome so that the pre-HSV-1 vector comprises a genome having less than 130 kbp and greater than 75 kbp. These deletions create genomic space allowing the introduction and delivery of very large foreign pieces of DNA. Introduction of a GAD expression cassette into the LAT region increase the genome size (i.e., number of base pairs) in the resulting HSV-1 vector.

As used herein, the term, one or more exogenous genes of interest can include, but are not limited to, a reporter gene (e.g., GFP, RFP, luciferase, or fused protein, etc.) driven by a transient promoter serving as internal expression control or for biodistribution studies; recombinases driven by an inducible promoter to allow in vivo modifying cellular or viral genes; antibiotic resistance genes such as chloramphenicol; elements of the Tetracycline inducible system (TRE); a GAD gene, or combinations thereof. The use of reporter genes such as, but not limited to, cherry, RFP, GFP, or CFP, can facilitate the identification of the recombined genome and to score both infectious particles (PFU) and transducing units (TU).

In some embodiments, the modified HSV-1 vector may optionally comprise a DNA sequence, for example an endogenous and/or foreign DNA sequence encoding a cell targeting protein, introduced into a genic region within the genome. The cell targeting protein can retarget virus entry into any tissue or cell type of interest. Cell targeting genes for insertion in HSV-1 vector used in the invention can include, but are not limited to, HER-2, IL13a2 or modified versions thereof. Examples of HSV modified glycoprotein D can be found, for example, in EP3469071, which is incorporated herein by reference.

### Promoter

Within a GAD expression cassette inserted into the viral vector as described herein, there is at least a promoter sequence, wherein the expression of the polynucleotide encoding GAD can be controlled by the promoter.

The promoter may comprise DNA sequence starting at least 2 kb, preferably 3 kb, more preferably 4 kb upstream to the initiation site of the polynucleotide encoding GAD. These sequences preferably contain known promoters' sequence elements, such as specific transcription binding sites, and distal sequences upstream of the gene, containing additional regulatory elements.

The promoter useful in the invention can be any promoter desired to control/regulate the expression of a polynucleotide encoding GAD. Exemplary promoters useful in the methods and treatment regimens of this application include, but are not limited to, human ubiquitin promoters and human synapsing promoters. Also, other known tissue-specific or cell-specific promoters may be used.

In some embodiments, the promoters contemplated is a constitutive mammalian promoter, such as are known in the art (e.g., EF-1α, UBC, β-actin, PGK, and the like).

In some embodiments, promoters useful in the invention may be active selectively in afferent neurons. By “active selectively in afferent neurons” it is meant herein that the promoter is active mainly or only in the afferent neurons and drives transcription of the RNA. Promoters for use in afferent neurons can be selected from, but are not limited to, promoters of genes coding for sensory neuroreceptors such as Transient Receptor Potential Vanilloid 1 (TRPV1) or Transient Receptor Potential cation channel subfamily M member 8 (TRPM8); and promoters of genes coding for sensory neuromodulators or sensory neurotransmitters, such as the promoters of Substance P, PACAP, Calcitonin Gene Related Peptide (CGRP). In some embodiments, promoter of genes coding for sensory neuroreceptors according to the invention is a promoter of the TRP gene family, more preferentially the promoter TRPV1 or TRPM8. In some embodiments, promoters of genes coding for sensory neuromodulators or sensory neurotransmitters according to the invention is the CGRP, or the promoter of genes involved in neurite outgrowth and stress response in sensory neurons, preferably the promoter of the gene encoding advillin (ADVL). In other embodiments, the promoter within a GAD expression cassette inserted into the viral vector as described herein can be an inducible promoter.

Also, those of skill in the art will recognize that many such mammalian afferent neuron specific promoters are known, and additional afferent neuron specific promoters are continually being discovered. All such afferent neuron specific promoters are encompassed by the present invention. As a nonlimiting example, the afferent neuron specific promoters contemplated can be selected from those disclosed in WO2017220800A1 and in Joussain et al. Int. J. Mol. Sci. 2022, 23, 8474.

In some embodiments, the promoter useful in the invention can be a ubiquitous promoter, such as, without limitation, human cytomegalovirus (HCMV) promoter, human elongation factor 1α (hEF-1α) promoter, β-actin promoter, rous sarcoma virus (RSV) promoter, human ubiquitin C (hUBC) promoter, ubiquitin B promoter, simian vacuolating virus 40 (SV40) promoter, phosphoglycerate kinase (PGK) promoter, β-globin promoter, NF-κB promoter, EGR1 promoter, elF4A1 promoter, FerL promoter, GAPDH promoter, β-Kin promoter, ROSA26 promoter, and human surfactant protein C (hSP-C) promoter. By “ubiquitous promoters” or “non-specific promoters” it is meant herein that the promoters are active in a wide range of cells, tissues, and/or organs.

In some embodiments, non-specific promoter according to the invention is selected from the hEF-1a promoter of SEQ ID NO: 5, the HCMV promoter of SEQ ID NO: 6, the RSV promoter of SEQ ID NO: 7, the hUBC promoter of, the SV40 promoter of SEQ ID NO: 9, the PGK promoter of SEQ ID NO: 10, the β-globin promoter, the NF-κB promoter, and the hSP-C promoter.

In a preferable embodiment, non-specific promoter useful in the method is the hEF-1α promoter (SEQ ID NO: 5).

The viral expression vector of the invention is directed more particularly to vertebrate, preferably to mammals, more preferably primates and humans. Therefore, those skilled in the art will recognize that such promoters are specific to species and would be able to select homologous sequences of a particular species of interest. For example, the promoters according to the invention are human homolog of rat TRPV1 or human TRPM8, or rat CGRP, or human CGRP, or rat advillin of or human advillin, amongst others.

### Dermatome

A “dermatome” is an area of skin that is mainly supplied by afferent nerve fibers from the dorsal root of any given spinal nerve. In other words, a dermatome is a distinct area of skin defined by its connection to one of 30 spinal nerves. There are 8 cervical nerves (C1-C8, C1 being an exception with no dermatome), 12 thoracic nerves (T1-T12), 5 lumbar nerves (L1-L5), and 5 sacral nerves (S1-S5). Each of these nerves relays sensation (including pain) from a particular region of skin (i.e., a particular dermatome) to the brain. Dermatomes are clinically important as they can help to diagnose a variety of conditions. For example, symptoms that occur along a specific dermatome may indicate a condition associated with a specific spinal nerve.

In some embodiments, in order to treat spasticity, the viral vector for upregulating GAD genes as described herein can be administered by one or more injections into one or more dermatomes. In some embodiments, in order to treat spasticity, the viral vector for upregulating GAD genes as described herein can be administered by multiple injections, preferably via subcutaneous inoculation, into multiple dermatomes. Multiple injections may be necessary to recruit as many afferent nerves as possible to achieve the sufficient expression of GAD gene. In some embodiments, in order to treat spasticity, the viral vector for upregulating GAD genes as described herein can be administered by 1-30 injections, preferably via subcutaneous inoculation, into multiple dermatomes. In some embodiments, in order to treat spasticity, the viral vector for upregulating GAD genes as described herein can be administered by 5-20 injections, preferably via subcutaneous inoculation, into multiple dermatomes. In some embodiments, in order to treat spasticity, the viral vector for upregulating GAD genes as described herein can be administered by 10-15 injections, preferably via subcutaneous inoculation, into multiple dermatomes. In some embodiments, in order to treat spasticity, the viral vector for upregulating GAD genes as described herein can be administered by 10, 11, 12, 13, 14, 15, 16, 17, 18, 19, or 20 injections, preferably via subcutaneous inoculation, into multiple dermatomes. In some embodiments, in order to treat spasticity, the viral vector for upregulating GAD genes as described herein can be administered by 10, 11, 12, 13, 14, 15, 16, 17, 18, 19, or 20 injections, preferably via subcutaneous inoculation, into 1, 2, 3, 4, 5, 6, 7, 8, 9, or 10 dermatomes, wherein each dermatome receives 1 to 20 injections. In some embodiments, the dermatomes into which the viral vectors for upregulating GAD genes are injected can be determined according to the spastic muscles that the viral vectors target to treat.

In some embodiments, in order to treat spasticity, the viral vector for upregulating GAD genes as described herein can be administered by multiple injections, preferably via subcutaneous inoculation, into one dermatome. In some embodiments, in order to treat spasticity, the viral vector for upregulating GAD genes as described herein can be administered by multipoint subcutaneous injection into one dermatome. In some embodiments, the multipoint subcutaneous injections contemplated include 1-20 injections at 1-20 sites of a target dermatome, respectively, to cover the entire or most surface of the target dermatome. In some embodiments, the multipoint subcutaneous injections contemplated include 5-15 injections at 5-15 sites of a target dermatome, respectively, to cover the entire or most surface of the target dermatome. In some embodiments, the multipoint subcutaneous injections contemplated include 5, 6, 7, 8, 9, 10, 11, 12, 13, 14 or 15 injections at 5, 6, 7, 8, 9, 10, 11, 12, 13, 14 or 15 sites of a target dermatome, respectively, to cover the entire or most surface of the target dermatome.

In some embodiments, a medical device for administrating multipoint subcutaneous injections can be used to provide multiple injections of the viral vectors as described herein at once at multiple sites of a target dermatome to cover the entire or most surface of the target dermatome. In some embodiments, the medical device can provide 1-10 injections of the viral vectors as described herein at once at multiple sites of a target dermatome to cover at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90% surface of the target dermatome. In some embodiments, the medical device can provide 2-9 injections of the viral vectors as described herein at once at multiple sites of a target dermatome to cover the entire or most surface of the target dermatome. In some embodiments, the medical device can provide 3, 4, 5, 6, 7, or 8 injections of the viral vectors as described herein at once at multiple sites of a target dermatome to cover the entire or most surface of the target dermatome. The medical device for administrating multipoint subcutaneous injections may include any medical devices, known or continually being discovered, that are capable of providing subcutaneous injections at multiple sites in a minimally invasive way (e.g., Multi-Injectors, Circular, 7-needle connections, Mesoram®). In a preferred embodiment, the viral vector for upregulating GAD genes is a defective viral vector derived from HSV as described herein. Depending on the severity of spasticity or particular spastic muscle, in some embodiments, one injection into one particular dermatome is required to be therapeutically effective; in some embodiments, multiple injections into one particular dermatome over a certain period of time are required to be therapeutically effective; In some embodiments, multiple injections into each of multiple particular dermatomes, whether adjacent to or several dermatomes away from each other, are required to be therapeutically effective. Following injection into the dermatomes, the viral vectors, such as vector derived from HSV-1, will reach the sensory DRG innervating the dermatomes from where they will stably express the therapeutic transgene, provided that adequate promoters drive their expression. The therapeutic transgene as described herein can be a polynucleotide encoding a wild type or modified GAD or an active fragment thereof, such as a wild type or modified GAD67 or an active fragment thereof, or a wild type or modified GAD65 or an active fragment thereof.

### Pharmaceutical Composition

The viral vector as described in this application can be administered in a pharmaceutical composition which may comprise a pharmaceutically acceptable carrier. The carrier of the composition can be any suitable carrier for the vector. The carrier typically will be liquid, but also can be solid, or a combination of liquid and solid components. The carrier desirably is a pharmaceutically acceptable (e.g., a physiologically or pharmacologically acceptable) carrier (e.g., excipient or diluent). The composition can further comprise any other suitable components, especially for enhancing the stability of the composition and/or its end-use. Accordingly, there is a wide variety of suitable formulations of the composition of the viral vector. The following formulations and methods are merely exemplary and are in no way limiting.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the viral vector and physiological distress. Immune system suppressors can be administered with the composition to reduce any immune response to the vector itself or associated with a disorder. Alternatively, immune enhancers can be included in the composition to upregulate the body's natural defenses against disease. Antibiotics, i.e., microbicides and fungicides, can be present to reduce the risk of infection associated with gene transfer procedures and other disorders.

The method of treating spasticity further can comprise the administration (i.e., pre-administration, co-administration, and/or post-administration) of other treatments and/or agents to modify (e.g., enhance) the effectiveness of the method. The method of the invention can further comprise the administration of other substances which locally or systemically alter (i.e., diminish or enhance) the effect of the composition on a host. For example, substances that diminish any systemic effect of the protein produced through expression of the nucleic acid sequence of the vector in a host can be used to control the level of systemic toxicity in the host. Likewise, substances that enhance the local effect of the protein produced through expression of the nucleic acid sequence of the vector in a host can be used to reduce the level of the protein required to produce a prophylactic or therapeutic effect in the host. Such substances include antagonists, for example, soluble receptors or antibodies directed against the protein produced through expression of the nucleic acid sequence of the vector, and agonists of the protein.

### Dermatome-Specific Administration

In some embodiments, also provided in this application is a method of administering the viral vector to a subject for the treatment of spasticity.

As used herein, the term “administration” or “administering” is defined to include an act of providing a pharmaceutical composition of the viral vector as described herein to a subject in performing the methods of the invention. Exemplary routes of administration include, but are not limited to, intravenously, intraarticularly, intracisternally, intraocularly, intraventricularly, intrathecally, subcutaneously, subpially, intramuscularly, intraperitoneally, intradermally, intracavitarily, and the like, as well as combinations of any two or more thereof. In some embodiments, the defective viral vector derived from HSV as described herein may be delivered directly into the spinal parenchyma, intrathecal space of the spine, into the spinal subpial space of the subject, and/or into the peripheral spastic muscle to achieve spinal upregulation of the GAD genes. (See, e.g., WO2016/122791.) In some embodiments, the defective viral vector derived from HSV as described herein may be delivered peripherally into any skin area to transfect the connected afferent neurons. (See, e.g., Liu et al., MOLECULAR THERAPY, 2004, Vol. 10, No. 1, 57-66.)

The term “therapeutically effective amount” or “effective amount” means the amount of the viral vectors that will elicit the biological or medical response of a tissue, system, animal or human that is being sought by the researcher, veterinarian, medical doctor or other clinician, e.g., the upregulation of the GAD genes in the afferent nerve fibers supplying a dermatome, including GAD67 gene and GAD65 gene, preferably GAD67 gene. Thus, the term “therapeutically effective amount” is used herein to denote any amount of a formulation that causes a substantial improvement in a condition associated with spasticity when applied to the affected areas repeatedly over a period of time. The amount will vary with the condition being treated, the stage of advancement of the condition, and the type and concentration of formulation applied.

Determining a therapeutically or prophylactically effective amount of the delivery vector can be done based on animal data using routine computational methods. Appropriate doses will depend, among other factors, on the specifics of the transfer vector chosen, on the route of administration, on the number of injection sites, on the mammal being treated (e.g., human or non-human primate or other mammal), age, weight, and general condition of the subject to be treated, the severity of the disorder being treated, the location of the area within the heart being treated and the mode of administration. Thus, the appropriate dosage may vary from patient to patient.

Dosage treatment may be a single dose schedule or a multiple dose schedule. Moreover, the subject may be administered as many doses as appropriate. The dosage may need to be adjusted to take into consideration an alternative route of administration, decrease in expression efficacy over time, or to balance the therapeutic benefit against any side effects.

HSV-1 vectors can efficiently infect cells and resist immune clearance, which might be attributed to the innate immune-evasive properties of HSV tegument proteins. Natural immune-evading functions coupled with deletion of IE genes from the HSV-1 vector backbone (such as ICP4, ICP22, and ICP27) allow multiple doses of the defective viral vector derived from HSV-1 vectors to improve transduction efficiency and make the vector especially well-suited for gene therapy. (See Heldwein et al., Cell. Mol. Life Sci., 2008, 65, 1653-1668. Tognarelli et al., Front. Cell. Infect. Microbiol., 2019, 9, 127. Yang et al., Front. Immunol., 2019, 10, 2196. Gurevich et al., Nature Medicine, 2022, 28, 780-788.)

Provided herein are also methods of treating spasticity in a subject comprising administrating multiple doses of the HSV-1 vectors as described herein to the subject. Via the course of treatment of spasticity, multiple doses of the HSV-1 vectors as described herein can be administered based on the severity of spasticity symptoms, with 1-10 doses being typically administered, at intervals from about 21 days (three weeks) to about three years. Intervals from about 3 months to about 12 months can be employed if, for example, it is necessary to modify the treatment schedule to improve therapeutic effects or to reduce side-effects. Over the course of treatment of the spasticity, 2-10 treatments (each treatment, e.g., includes one or more subcutaneous injections at once, with or without a multi-injection device as described herein) of the vectors as described herein can be administered, with 2-4 treatments being typically administered, at intervals of about 3, about 6, about 12, about 24, or about 36 months. In some embodiments, the vectors as described herein can be administered to a subject at a dose of from about 1× 10⁶ vg/kg to about 1×10¹⁵ vg/kg of body weight. In some embodiments, the vectors as described herein can be administered to a subject at a dose of from about 1×10⁷ vg/kg to about 1×10¹² vg/kg of body weight. Over the course of treatment of the spasticity, the dose can be constant over time, or the dose may decrease or increase over time to optimize the therapeutic effects and to minimize side effects.

Optionally, HSV-1-mediated delivery according to the invention may be combined with delivery by other viral and non-viral vectors. Such other viral vectors may include, without limitation, adenoviral vectors, adeno-associated virus (AAV) vectors, retroviral vectors, lentiviral vectors, and baculovirus vectors. Non-viral vectors may include, without limitation, liposomes, lipid-based vectors, polyplex vectors, molecular conjugates, polyamines and polycation vectors.

In a preferred embodiment, this application provides a method of treating spasticity in a subject comprising upregulating GAD gene, thereby treating spasticity in the subject. The upregulation of GAD gene comprises administrating to the subject a viral vector comprising a polynucleotide encoding GAD, wherein GAD is expressed, thereby decreasing spasticity. The viral vector is the defective viral vector derived from HSV as described herein. The polynucleotide may encode a wild type or modified GAD67 and/or a wild type or modified GAD65 or an active fragment thereof, more preferably a wild type or modified GAD67 or an active fragment thereof. The defective viral vector derived from HSV as described herein is administered directly into one or more dermatomes of the subject. In one aspect, the defective viral vector derived from HSV as described herein is administered directly into one or more dermatomes of the subject by subcutaneous inoculation. As described herein, depending on the severity of spasticity or the particular spastic muscle, in some embodiments, one injection of the vector into one particular dermatome is required to be therapeutically effective; in some embodiments, multiple injections of the vector into one particular dermatome over a certain period of time are required to be therapeutically effective. In some embodiments, multiple injections of the vector into each of multiple particular dermatomes, whether adjacent to or several dermatomes away from each other, are required to be therapeutically effective. In some embodiments, the dermatome selected for administration of the viral vector is connected with the spastic muscle group. Administrating the viral vectors to the dermatome allows infection of sensory neurons associated with the spastic muscle group, causing the sensory neurons to produce and release GABA and to transform into inhibitory neurons, thereby reducing spasticity.

In a preferred embodiment, a method of treating spasticity in a subject comprises administrating to the subject a therapeutically effective amount of a viral vector comprising a polynucleotide encoding GAD, thereby treating spasticity in the subject. The viral vector is the defective viral vector derived from HSV as described herein. The polynucleotide may encode a wild type or modified GAD67 and/or a wild type or modified GAD65 or an active fragment thereof, more preferably a wild type or modified GAD67 or an active fragment thereof. The defective viral vector derived from HSV as described herein is administered directly into one or more dermatomes of the subject. In one aspect, the defective viral vector derived from HSV as described herein is administered directly into one or more dermatomes of the subject by subcutaneous inoculation. As described herein, depending on the severity of spasticity or the particular spastic muscle, one or more injections of the vector into one or more dermatomes may be required to be therapeutically effective.

In another aspect, the invention also provides a treatment regimen for treating a subject suffering spasticity or a condition associated with spasticity. The treatment regimen includes administering an afferent neuron-specific upregulation of the GAD genes, including GAD67 gene and GAD65 gene, preferably GAD67 gene. As discussed in detail above, upregulation of GAD (preferably GAD67) may include administering a viral vector encoding GAD gene (preferably GAD67 gene), wherein GAD (preferably GAD67) is expressed, causing the neuron to produce and release GABA, i.e., converting the excitatory neurotransmitters to inhibitory neurotransmitters, and treats the spasticity or a condition associated with spasticity. As described herein, depending on the severity of spasticity or the particular spastic muscle, one or more injections of the vector into one or more dermatomes may be required to be therapeutically effective.

### Mechanism of Action

In some embodiments, this application proposes a mechanism of action for using the viral vector as described herein (preferably, a defective viral vector derived from HSV-1) for the treatment of spasticity. The method of treating spasticity as disclosed is region-specific (i.e., specifically acts in spinal micro circuitries that are producing abnormal muscle activity while avoiding broad dampening of spinal functions (or even other brain functions as baclofen does)) and tailorable (e.g., be able to modulate neural activity based on the severity of spasticity symptoms) and provides long-term antispastic effects.

Injection of the viral vector as described herein into a dermatome will allow the vector to infect the afferent nerve fibers connected to the dermatome, leading to the stable expression of GAD genes (preferably GAD67 gene) in sensory neurons driven by the promoters as described herein. GAD (preferably GAD67) functions as an enzyme inducing and facilitating the synthesis of the inhibitory neurotransmitter GABA from the excitatory neurotransmitter glutamate. Sensory neurons infected by the vector will be capable of producing and releasing GABA at an increased level. The muscle excitability is thus reduced, and spasticity is treated or alleviated.

The novel combination of the HSV-1 mediated gene therapy upregulating GAD gene and the dermatome-specific administration allows to target a designated spinal level associated with a particular dermatome and thus play a therapeutic role on the specific spastic muscle. Due to the neurotropic nature of HSV-1 vector and through the dermatome-specific administration, the therapeutically effective amount required for treating spasticity will be significantly reduced and the systemic toxicity and side effects will be minimized. This invention provides a nonsurgical, minimally invasive, region-specific, therapeutic approach to treat spasticity.

The following examples are intended to illustrate but not limit the invention. All citations throughout the disclosure are hereby expressly incorporated by reference.

### Examples

Experiments can be carried out to evaluate the effects of the method as disclosed on in vivo conversion of glutamatergic sensory neurons into GABAergic neurons, to modulate the hyper-excitability of the spinal circuitries and thus to treat spasticity.

HSV-GAD67 viral vectors can be directly injected into a mouse model of chronic sacral SCI to infect sensory afferent neurons to reduce spasticity. Behavioral effects of direct injection on the mouse model can be evaluated and compared with the effects of Baclofen. Anatomical assessment can be performed to further evaluate the effects of the direct injection of the HSV vectors as described herein is an effective and less invasive therapeutic approach to treat spasticity.

Neuromodulatory HSV-Mediated Gene Therapy to Treat Spasticity after Spinal Cord Injury

The following experiments were performed to determine the effects of in vivo conversion of glutamatergic sensory neurons into GABAergic neurons to modulate the hyper-excitability of the spinal circuitries, typical of spasticity.

In the following experiments, all mice (n=20) had a complete spinal cord transection at the sacral level between S1 and S2 level. Such animals develop a spastic phenotype of the tail. More specifically, their tail shows an abnormal posture with an increased tonic stretch reflex and spontaneous or induced spasms (related to an increased phasic reflex).

The mice were randomly assigned into either the Treated group or the Control group (n=10 each) 8 weeks after spinal transection to allow the spastic phenotype to develop. The “Treated” group was injected subcutaneously 20 microliters in 6 points distributed around the base of the tail, with a non-replicative HSV-1 vector expressing GAD67 (described below), the major enzyme converting glutamate into GABA. The “Control” group was injected in the same way with a UV-inactivated product. The injection points at the base of the tail are into the dermatome of the sacral region.

Vector description. The vector (referred to herein as hEF-1α::GAD67) is a non-replicative recombinant herpes simplex virus type 1 (HSV-1)-derived vector that expresses the human glutamate decarboxylase (hGAD67) driven by the human elongation factor 1 alpha (hEF-1α) promoter. The transcription cassette was inserted into the latency-associated transcript locus of the HSV-1 genome. The vector was deleted in the immediate early (IE) viral genes for ICP4 and ICP27 to block its ability to replicate. In addition, one copy of the ICP0 gene was also deleted to reduce the toxicity of the vector. The hEF-1α::GAD67 vector is provided as purified viral particles in phosphate buffered saline (PBS) at a concentration of 1.3×10⁵ PFU (Plaque Forming Unit)/μl (titration by plaque assay on cells in monolayer).

### Assessment of Treatment on Tonic Activity by Tail Posture Characterization

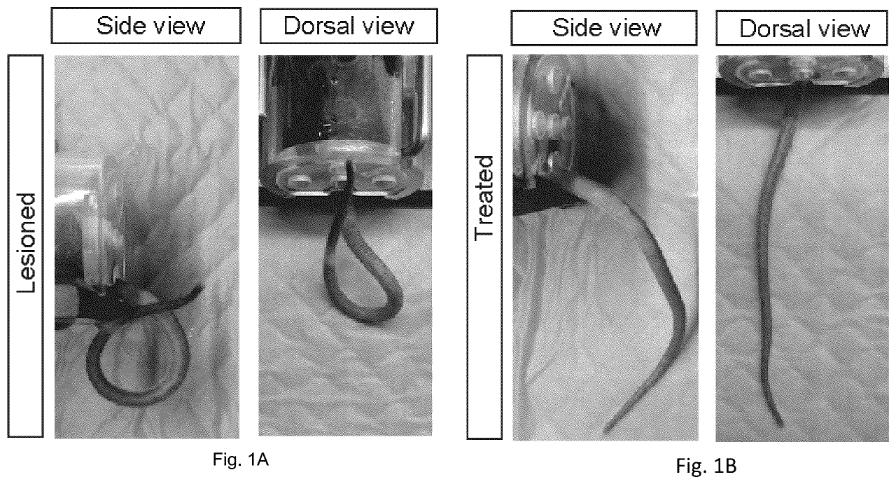

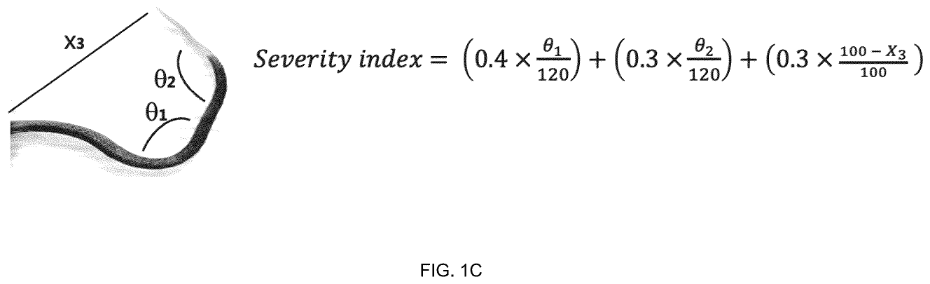

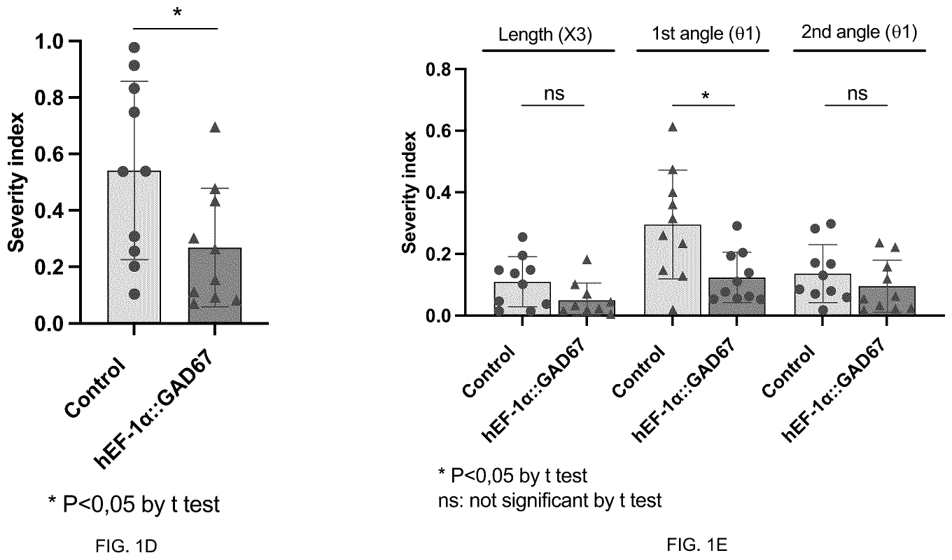

As described in Marcantoni et al. (Sci. Transl. Med. 12, eaay0167 (2020)), a severity index characterizing the increase of the tonic stretch exaggeration leading to abnormal posture quantification has been defined for this model (FIG. 1C). FIGS. 1A and 1B shows representative images of mouse tails in lesioned control animals (FIG. 1A) and treated animals (FIG. 1B) in the chronic state of SCI, 3 weeks after the injection of the vectors. FIG. 1D shows severity index of each group (N=10 animals per group). Each dot represents one animal and bar graphs represent mean±standard deviation. Statistical analysis with unpaired t-Test shows a significant difference (p<0.05) between the treated mice compared to the lesioned control ones, where the severity index is significantly decreased after hEF1α::GAD67 treatment. When splitting the global index into its 3 contributors (FIG. 1E), only one, the first curvature of the tail 01, shows statistically significant difference between the groups. These results show a local effect on the first muscle segment (proximal to the tail base) of the hEF-1α::GAD67 vector. Therefore, a statistically significant reduction of the tonic muscle hyperactivity is demonstrated when mice are treated with hEF-1α::GAD67 vector.

### Electromyography Evaluation of Vector Effect on Spasms of the Tail

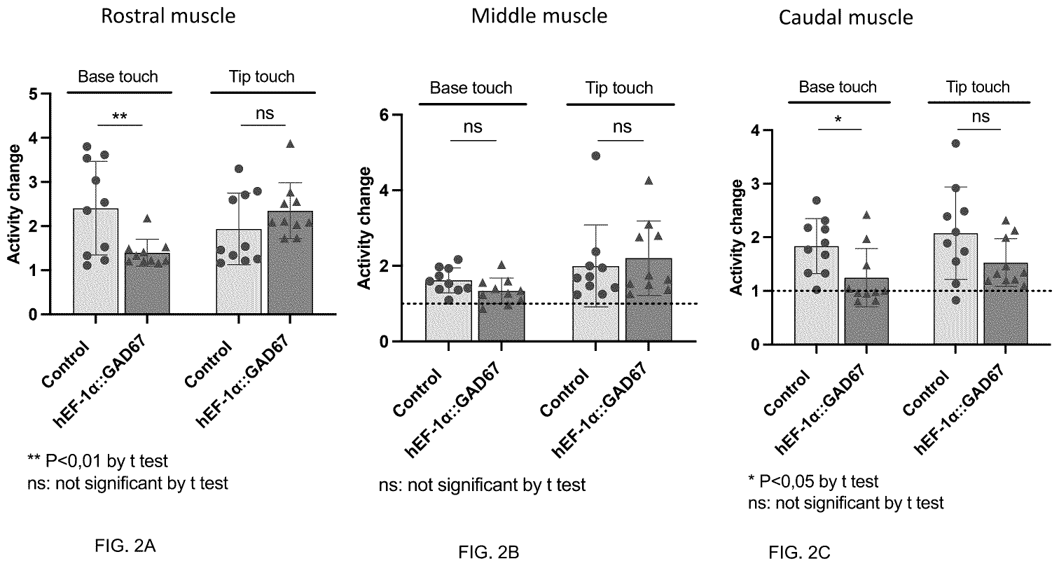

In lesioned mice, in a similar way as in human, spasms can occur in muscles below the lesion either spontaneously or induced by sensory stimuli. To evaluate the effect of the vector on such spasms, electromyography (EMG) was used to measure muscle activity. Animals were evaluated at least 3 weeks after injection (3 to 13 weeks post-injection) in order to allow the vector to reach a stable pseudo-latent stage in infected cells. Area under curve was calculated for each EMG recording of 2 seconds with 10 repeats for each animal, either without any stimulation (animal was at rest without any contact on the tail except the EMG electrodes) or during tactile stimulation of the skin. Tactile stimulation was implemented at 2 different levels: base of the tail, where the vectors were previously injected, and tip of the tail. FIG. 2 shows for each animal, mean EMG change upon stimulation (tip or base) compared to baseline without stimulation. FIG. 2 (A) shows EMG recording of the muscle at the base of the tail, allegedly related to the same metameric level as the injected dermatome (S1-S2). Control group shows an increase of activity with both Tip and Base stimuli. Treated group shows an increased activity upon stimulation of the tip of the tail (not statistically significant from the control group) but shows a statistically significant (p<0.05-2way Anova with Tukey's multiple comparisons) reduced activity upon stimulation of the base of the tail. Therefore, hEF-1α::GAD67 vector has an effect in limiting the spasms induced in the tail upon stimulation, and that this activity seems to be localized upon stimulation of infected neurons.

To further investigate the effect of the hEF-1α::GAD67 vector, 2 other tail muscles were assessed with similar EMG recording at the same time as the base muscle: one in the middle of the tail, and one at the tip (more caudal) of the tail, which are believed to be primarily driven by more caudal metameric levels. No statistically significant effect on spasm intensity has been achieved, suggesting that the effect of the vector may be limited to the muscle activity driven by the same metameric level as the injected dermatome (see FIGS. 2B and 2C).

EMG Evaluation on Spasms of the Tail of a Combination of the Vector with Tiagabine.

Next it was determined whether the effect of overexpression of GAD67 leading to increase of GABA release at the spinal level could be improved by GABA re-uptake inhibitor like tiagabine. The same set of EMG evaluation was conducted with Control and Treated groups also both injected with 15 mg/kg tiagabine before evaluation.

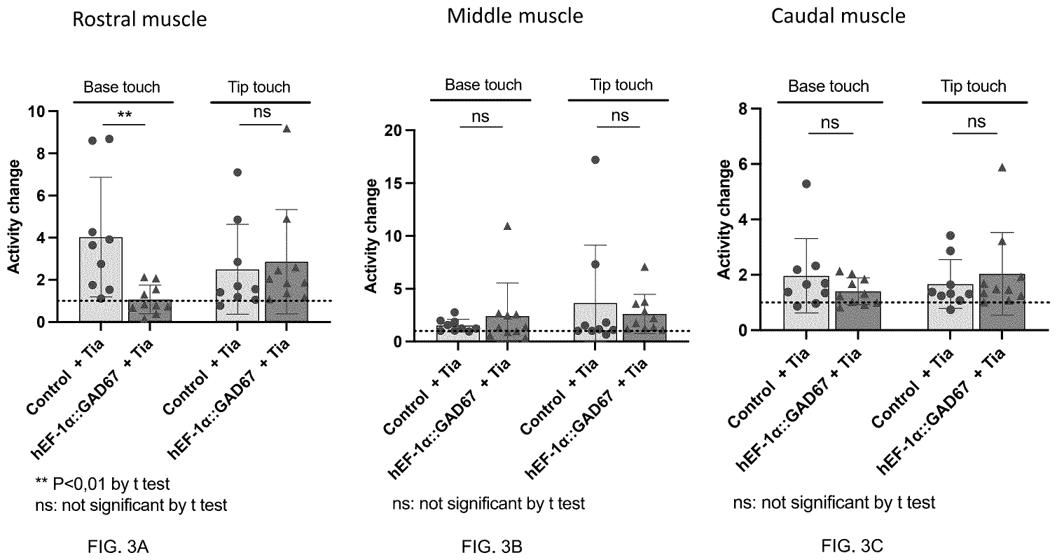

FIG. 3A shows the change of EMG activity of base tail muscle upon tactile stimulation at base and tip of the tail. As observed in the treatment without tiagabine above, only the Treated group shows absence of increased EMG activity, hence absence of induced spasms of base tail muscle upon tactile stimulation of the base of the tail only. Of note the difference between Treated and Control group is highly significant (p<0.001-2way Anova with Tukey's multiple comparisons). As seen from the activity induced by tip tail stimulation, tiagabine does not have an effect alone to prevent spasms. Additionally, in 5 out of 10 animals, the stimulation of the base of the tail induces a reduction of the muscle activity. Such experiment confirms the modulatory effect of tiagabine in combination with the vector.

In presence of tiagabine, the muscle activity of middle and caudal muscle of the tail was evaluated after base stimulus (FIGS. 3B and 3C) and no statistically significant difference was observed on induced muscle activity compared to Control animals.

### CONCLUSION

Overall, these experiments support the conclusion that local subcutaneous injection of hEF-1α::GAD67 vector has a local effect on muscle tone and on muscle spasms, that can be synergistically increased by tiagabine.

Without wishing to be bound to any particular theory, it is believed that the sensory nerves of the injected dermatome, infected by the vector, release GABA from their axon endings at the spinal level, instead of only glutamate. Such release of GABA is believed to have an impact on the overall excitability of the motoneurons that are known to be hyperactive in spasticity.

Tactile stimulation of the skin at the site of injection, inducing sensory nerve firing, shows a local muscle activity reduction instead of the well-known spasm induction. The impact seems to be limited to the muscle which activity is primarily driven by the same metameric level as the injected dermatome, suggesting that GABA release at the spinal level may not have spread effect.

SEQ ID NO: 1
3610
DNA
Human glutamate decarboxylase 67
ATGGCGTCTTCGACCCCATCTTCGTCCGCAACCTCCTCGAACGCGGGAGCGGACCCC
AATACCACTAACCTGCGCCCCACAACGTACGATACCTGGTGCGGCGTGGCCCATGG
ATGCACCAGAAAACTGGGGCTCAAGATCTGCGGCTTCTTGCAAAGGACCAACAGCC
TGGAAGAGAAGAGTCGCCTTGTGAGTGCCTTCAGGGAGAGGCAATCCTCCAAGAAC
CTGCTTTCCTGTGAAAACAGCGACCGGGATGCCCGCTTCCGGCGCACAGAGACTGA
CTTCTCTAATCTGTTTGCTAGAGATCTGCTTCCGGCTAAGAACGGTGAGGAGCAAAC
CGTGCAATTCCTCCTGGAAGTGGTGGACATACTCCTCAACTATGTCCGCAAGACATT
TGATCGCTCCACCAAGGTGCTGGACTTTCATCACCCACACCAGTTGCTGGAAGGCAT
GGAGGGCTTCAACTTGGAGCTCTCTGACCACCCCGAGTCCCTGGAGCAGATCCTGGT
TGACTGCAGAGACACCTTGAAGTATGGGGTTCGCACAGGTCATCCTCGATTTTTCAA
CCAGCTCTCCACTGGATTGGATATTATTGGCCTAGCTGGAGAATGGCTGACATCAAC
GGCCAATACCAACATGTTTACATATGAAATTGCACCAGTGTTTGTCCTCATGGAACA
AATAACACTTAAGAAGATGAGAGAGATAGTTGGATGGTCAAGTAAAGATGGTGATG
GGATATTTTCTCCTGGGGGCGCCATATCCAACATGTACAGCATCATGGCTGCTCGCT
ACAAGTACTTCCCGGAAGTTAAGACAAAGGGCATGGCGGCTGTGCCTAAACTGGTC
CTCTTCACCTCAGAACAGAGTCACTATTCCATAAAGAAAGCTGGGGCTGCACTTGGC
TTTGGAACTGACAATGTGATTTTGATAAAGTGCAATGAAAGGGGGAAAATAATTCC
AGCTGATTTTGAGGCAAAAATTCTTGAAGCCAAACAGAAGGGATATGTTCCCTTTTA
TGTCAATGCAACTGCTGGCACGACTGTTTATGGAGCTTTTGATCCGATACAAGAGAT
TGCAGATATATGTGAGAAATATAACCTTTGGTTGCATGTCGATGCTGCCTGGGGAGG
TGGGCTGCTCATGTCCAGGAAGCACCGCCATAAACTCAACGGCATAGAAAGGGCCA
ACTCAGTCACCTGGAACCCTCACAAGATGATGGGCGTGCTGTTGCAGTGCTCTGCCA
TTCTCGTCAAGGAAAAGGGTATACTCCAAGGATGCAACCAGATGTGTGCAGGATAT
CTCTTCCAGCCAGACAAGCAGTATGATGTCTCCTACGACACCGGGGACAAGGCAAT
TCAGTGTGGCCGCCACGTGGATATCTTCAAGTTCTGGCTGATGTGGAAAGCAAAGGG
CACAGTGGGATTTGAAAACCAGATCAACAAATGCCTGGAACTGGCTGAATACCTCT
ATGCCAAGATTAAAAACAGAGAAGAATTTGAGATGGTTTTCAATGGCGAGCCTGAG
CACACAAACGTCTGTTTTTGGTATATTCCACAAAGCCTCAGGGGTGTGCCAGACAGC
CCTCAACGACGGGAAAAGCTACACAAGGTGGCTCCAAAAATCAAAGCCCTGATGAT
GGAGTCAGGTACGACCATGGTTGGCTACCAGCCCCAAGGGGACAAGGCCAACTTCT
TCCGGATGGTCATCTCCAACCCAGCCGCTACCCAGTCTGACATTGACTTCCTCATTG
AGGAGATAGAAAGACTGGGCCAGGATCTGTAA
SEQ ID NO: 2
594
AA
Human glutamate decarboxylase 67
MASSTPSSSATSSNAGADPNTTNLRPTTYDTWCGVAHGCTRKLGLKICGFLQRINSLEE
KSRLVSAFRERQSSKNLLSCENSDRDARFRRTETDFSNLFARDLLPAKNGEEQTVQFLLE
VVDILLNYVRKTFDRSTKVLDFHHPHQLLEGMEGFNLELSDHPESLEQILVDCRDTLKY
GVRTGHPRFFNQLSTGLDIIGLAGEWLTSTANTNMFTYEIAPVFVLMEQITLKKMREIVG
WSSKDGDGIFSPGGAISNMYSIMAARYKYFPEVKTKGMAAVPKLVLFTSEQSHYSIKKA
GAALGFGTDNVILIKCNERGKIIPADFEAKILEAKQKGYVPFYVNATAGTTVYGAFDPIQ
EIADICEKYNLWLHVDAAWGGGLLMSRKHRHKLNGIERANSVTWNPHKMMGVLLQCS
AILVKEKGILQGCNQMCAGYLFQPDKQYDVSYDTGDKAIQCGRHVDIFKFWLMWKAK
GTVGFENQINKCLELAEYLYAKIKNREEFEMVFNGEPEHTNVCFWYIPQSLRGVPDSPQ
RREKLHKVAPKIKALMMESGTTMVGYQPQGDKANFFRMVISNPAATQSDIDFLIEEIER
LGQDL*
SEQ ID NO: 3
2400
DNA
Human glutamate decarboxylase 65
AGCTCGCCCGCAGCTCGCACTCGCAGGCGACCTGCTCCAGTCTCCAAAGCCGATGGC
ATCTCCGGGCTCTGGCTTTTGGTCTTTCGGGTCGGAAGATGGCTCTGGGGATTCCGA
GAATCCCGGCACAGCGCGAGCCTGGTGCCAAGTGGCTCAGAAGTTCACGGGCGGCA
TCGGAAACAAACTGTGCGCCCTGCTCTACGGAGACGCCGAGAAGCCGGCGGAGAGC
GGCGGGAGCCAACCCCCGCGGGCCGCCGCCCGGAAGGCCGCCTGCGCCTGCGACCA
GAAGCCCTGCAGCTGCTCCAAAGTGGATGTCAACTACGCGTTTCTCCATGCAACAGA
CCTGCTGCCGGCGTGTGATGGAGAAAGGCCCACTTTGGCGTTTCTGCAAGATGTTAT
GAACATTTTACTTCAGTATGTGGTGAAAAGTTTCGATAGATCAACCAAAGTGATTGA
TTTCCATTATCCTAATGAGCTTCTCCAAGAATATAATTGGGAATTGGCAGACCAACC
ACAAAATTTGGAGGAAATTTTGATGCATTGCCAAACAACTCTAAAATATGCAATTAA
AACAGGGCATCCTAGATACTTCAATCAACTTTCTACTGGTTTGGATATGGTTGGATT
AGCAGCAGACTGGCTGACATCAACAGCAAATACTAACATGTTCACCTATGAAATTG
CTCCAGTATTTGTGCTTTTGGAATATGTCACACTAAAGAAAATGAGAGAAATCATTG
GCTGGCCAGGGGGCTCTGGCGATGGGATATTTTCTCCCGGTGGCGCCATATCTAACA
TGTATGCCATGATGATCGCACGCTTTAAGATGTTCCCAGAAGTCAAGGAGAAAGGA
ATGGCTGCTCTTCCCAGGCTCATTGCCTTCACGTCTGAACATAGTCATTTTTCTCTCA
AGAAGGGAGCTGCAGCCTTAGGGATTGGAACAGACAGCGTGATTCTGATTAAATGT
GATGAGAGAGGGAAAATGATTCCATCTGATCTTGAAAGAAGGATTCTTGAAGCCAA
ACAGAAAGGGTTTGTTCCTTTCCTCGTGAGTGCCACAGCTGGAACCACCGTGTACGG
AGCATTTGACCCCCTCTTAGCTGTCGCTGACATTTGCAAAAAGTATAAGATCTGGAT
GCATGTGGATGCAGCTTGGGGTGGGGGATTACTGATGTCCCGAAAACACAAGTGGA
AACTGAGTGGCGTGGAGAGGGCCAACTCTGTGACGTGGAATCCACACAAGATGATG
GGAGTCCCTTTGCAGTGCTCTGCTCTCCTGGTTAGAGAAGAGGGATTGATGCAGAAT
TGCAACCAAATGCATGCCTCCTACCTCTTTCAGCAAGATAAACATTATGACCTGTCC
TATGACACTGGAGACAAGGCCTTACAGTGCGGACGCCACGTTGATGTTTTTAAACTA
TGGCTGATGTGGAGGGCAAAGGGGACTACCGGGTTTGAAGCGCATGTTGATAAATG
TTTGGAGTTGGCAGAGTATTTATACAACATCATAAAAAACCGAGAAGGATATGAGA
TGGTGTTTGATGGGAAGCCTCAGCACACAAATGTCTGCTTCTGGTACATTCCTCCAA
GCTTGCGTACTCTGGAAGACAATGAAGAGAGAATGAGTCGCCTCTCGAAGGTGGCT
CCAGTGATTAAAGCCAGAATGATGGAGTATGGAACCACAATGGTCAGCTACCAACC
CTTGGGAGACAAGGTCAATTTCTTCCGCATGGTCATCTCAAACCCAGCGGCAACTCA
CCAAGACATTGACTTCCTGATTGAAGAAATAGAACGCCTTGGACAAGATTTATAATA
ACCTTGCTCACCAAGCTGTTCCACTTCTCTAGGTAGACAATTAAGTIGTCACAAACT
GTGTGAATGTATTTGTAGTTTGTTCCAAAGTAAATCTATTTCTATATTGTGGTGTCAA
AGTAGAGTTTAAAAATTAAACAAAAAAGACATTGCTCCTTTTAAAAGTCCTTTCTTA
AGTTTAGAATACCTCTCTAAGAATTCGTGACAAAAGGCTATGTTCTAATCAATAAGG
AAAAGCTTAAAATTGTTATAAATACTTCCCTTACTTTTAATATAGTGTGCAAAGCAA
ACTTTATTTTCACTTCAGACTAGTAGGACTGAATAGTGCCAAATTGCCCCTGAATCA
TAAAAGGTTCTTTGGGGTGCAGTAAAAAGGACAAAGTAAATATAAAATATATGTTG
ACAATAAAAACTCTTGCCTTTTTCATAGTATTAGAAAAAAATTTCTAATTTACCTATA
GCAACATTTCAAATGTATTTAAATACATATAATTTTACAAAAGGAAAATATATATAT
TAAAAAAGATATCCTATTTTGTAACATATAGATTTTTATTTTATATAGGTTATACAAA
CTGCGGGGGCGGAATT
SEQ ID NO: 5
1179
DNA
hEF-1a promoter
ggctccggtgcccgtcagtgggcagagcgcacatcgcccacagtccccgagaagttggggggaggggtcggcaattgaaccggtgcct
agagaaggtggcgcggggtaaactgggaaagtgatgtcgtgtactggctccgcctttttcccgaggggggggagaaccgtatataagtg
cagtagtcgccgtgaacgttctttttcgcaacgggtttgccgccagaacacaggtaagtgccgtgtgtggttcccgcgggcctggcctcttta
cgggttatggcccttgcgtgccttgaattacttccacctggctgcagtacgtgattcttgatcccgagcttcgggttggaagtgggtgggaga
gttcgaggccttgcgcttaaggagccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctgcgacgctttttttctggcaagatagt
cttgtaaatgcgggccaagatctgcacactggtatttcggtttttggggccgcgggcggcgacggggcccgtgcgtcccagcgcacatgttcgg
cgaggcggggcctgcgagcgcggccaccgagaatcggacgggggtagtctcaagctggccggcctgctctggtgcctggcctegcgcc
gccgtgtatcgccccgccctgggcggcaaggctggcccggtcggcaccagttgcgtgagcggaaagatggccgcttcccggccctgct
gcagggagctcaaaatggaggacgcggcgctcgggagagcgggcgggtgagtcacccacacaaaggaaaagggcctttccgtcctca
gccgtcgcttcatgtgactccacggagtaccgggcgccgtccaggcacctcgattagttctcgagcttttggagtacgtcgtctttaggttgg
ggggaggggttttatgcgatggagtttccccacactgagtgggtggagactgaagttaggccagcttggcacttgatgtaattctccttggaa
tttgccctttttgagtttggatcttggttcattctcaagcctcagacagtggttcaaagtttttttcttccatttcaggtgtcgtga
SEQ ID NO: 6
508
DNA
cytomegalovirus promoter
CGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGTA
TCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATAG
CGGTTTGACTCACGGGGATTTCCAAGTCTCCACCCCATTGACGTCAATGGGAGTTTG
TTTTGGCACCAAAATCAACGGGACTTTCCAAAATGTCGTAACAACTCCGCCCCATTG
ACGCAAATGGGCGGTAGGCGTGTACGGTGGGAGGTCTATATAAGCAGAGCT
SEQ ID NO: 7
552
DNA
rous-sarcoma virus promoter
GGTGCACACCAATGTGGTGAATGGTCAAATGGCGTTTATTGTATCGAGCTAGGCACT
TAAATACAATTATCTCTGCAATGCGGTATTCAGTGGTTCGTCCAATCCATGTCAGAC
CCGTCTGTTGCCTTCCTAATAAGGCACGATCGTACCACCTTACTTCCACCAATCGGC
ATGCACGGTGCTTTTTCTCTCCTTGTAAGGCATGTTGCTAACTCATCGTTACCATGTT
GCAAGACTACAAGAGTATTGCATAAGACTACATTTCCCCCTCCCTATGCAAAAGCGA
AACTACTATATCCTGAGGGGACTCCTAACCGCGTACAACCGAAGCCCCGCTTTTCGC
CTAAACACACCCTAGTCCCCTCAGATACGCGTATATCTGGCCCGTACATCGCGAAGC
AGCGCAAAACGCCTAACCCTAAGCAGATTCTTCATGCAATTGTCGGTCAAGCCTTGC
CTTGTTGTAGCTTAAATTTTGCTCGCGCACTACTCAGCGACCTCCAACACACAAGCA
GGGAGCAG
SEQ ID NO: 8
1212
DNA
ubiquitin C promoter
ggcctccgcgccgggttttggcgcctcccgcgggcgcccccctcctcacggcgagcgctgccacgtcagacgaagggcgcagcgagc
gtcctgatccttccgcccggacgctcaggacagcggcccgctgctcataagactcggccttagaaccccagtatcagcagaaggacatttt
aggacgggacttgggtgactctagggcactggttttctttccagagagcggaacaggcgaggaaaagtagtcccttctcggcgattctgcg
gagggatctccgtggggcggtgaacgccgatgattatataaggacgcgccgggtgtggcacagctagttccgtcgcagccgggatttggg
tcgcggttcttgtttgtggatcgctgtgatcgtcacttggtgagtagcgggctgctgggctggccggggctttcgtggccgccgggccgctc
ggtgggacggaagcgtgtggagagaccgccaagggctgtagtctgggtccgcgagcaaggttgccctgaactgggggttggggggag
cgcagcaaaatggcggctgttcccgagtcttgaatggaagacgcttgtgaggcgggctgtgaggtcgttgaaacaaggtggggggcatg
gtgggcggcaagaacccaaggtcttgaggccttcgctaatgcgggaaagctcttattcgggtgagatgggctggggcaccatctggggac
cctgacgtgaagtttgtcactgactggagaactcggtttgtcgtctgttgcgggggcggcagttatggcggtgccgttgggcagtgcacccg
tacctttgggagcgcgcgccctcgtcgtgtcgtgacgtcacccgttctgttggcttataatgcagggtggggccacctgccggtaggtgtgc
ggtaggcttttctccgtcgcaggacgcagggttcgggcctagggtaggctctcctgaatcgacaggcgccggacctctggtgaggggagg
gataagtgaggcgtcagtttctttggtcggttttatgtacctatcttcttaagtagctgaagctccggttttgaactatgcgctcggggttggc
gagtgtgttttgtgaagttttttaggcaccttttgaaatgtaatcatttgggtcaatatgtaattttcagtgttagactagtaaattgtccgct
aaattctggccgtttttggcttttttgttagac
SEQ ID NO: 9
331
DNA
simian vacuolating virus 40 promoter
GTGTGTCAGTTAGGGTGTGGAAAGTCCCCAGGCTCCCCAGGCAGGCAGAAGTATGC
AAAGCATGCATCTCAATTAGTCAGCAACCAGGTGTGGAAAGTCCCCAGGCTCCCCA
GCAGGCAGAAGTATGCAAAGCATGCATCTCAATTAGTCAGCAACCATAGTCCCGCC
CCTAACTCCGCCCATCCCGCCCCTAACTCCGCCCAGTTCCGCCCATTCTCCGCCCCAT
GGCTGACTAATTTTTTTTATTTATGCAGAGGCCGAGGCCGCCTCTGCCTCTGAGCTAT
TCCAGAAGTAGTGAGGAGGCTTTTTTGGAGGCCTAGGCTTTTGCAAA
SEQ ID NO: 10
500
DNA
phosphoglycerate kinase promoter
GGGTAGGGGAGGCGCTTTTCCCAAGGCAGTCTGGAGCATGCGCTTTAGCAGCCCCG
CTGGGCACTTGGCGCTACACAAGTGGCCTCTGGCCTCGCACACATTCCACATCCACC
GGTAGGCGCCAACCGGCTCCGTTCTTTGGTGGCCCCTTCGCGCCACCTTCTACTCCTC
CCCTAGTCAGGAAGTTCCCCCCCGCCCCGCAGCTCGCGTCGTGCAGGACGTGACAA
ATGGAAGTAGCACGTCTCACTAGTCTCGTGCAGATGGACAGCACCGCTGAGCAATG
GAAGCGGGTAGGCCTTTGGGGCAGCGGCCAATAGCAGCTTTGCTCCTTCGCTTTCTG
GGCTCAGAGGCTGGGAAGGGGTGGGTCCGGGGGCGGGCTCAGGGGGGGGCTCAGG
GGCGGGGGGGGCGCCCGAAGGTCCTCCGGAGGCCCGGCATTCTGCACGCTTCAAAA
GCGCACGTCTGCCGCGCTGTTCTCCTCTTCCTCATCTCCGGGCCTTTCG

1. A method of treating spasticity in a subject comprising upregulating GAD (glutamic acid decarboxylase) gene, thereby treating spasticity in the subject.

2. The method of claim 1, wherein the upregulation of GAD gene is a region-specific upregulation of GAD gene.

3. The method of claim 1, wherein the upregulation of GAD gene comprises administrating to the subject a viral vector comprising a polynucleotide encoding GAD, wherein GAD is expressed, thereby decreasing spasticity.

4. (canceled)

5. The method of claim 1, wherein the viral vector is a defective viral vector derived from HSV comprising an expression cassette comprising a polynucleotide encoding GAD is inserted in the LAT (Latency Associated Transcripts) locus of the defective viral vector.

6. (canceled)

7. (canceled)

8. The method of claim 5, wherein the expression cassette comprises a promoter.

9. The method of claim 8, wherein the promoter is an afferent neuron-specific promoter selected from promoters of genes coding for sensory neuroreceptors, preferably a promoter of the TRP gene family, more preferably the promoter of TRPV1 or TRPM8; or promoters of genes coding for sensory neuromodulators or sensory neurotransmitters, preferably promoter of substance P, PACAP, CGRP, ADVL, more preferably promoter of CGRP or ADVL.

10. The method of claim 8, wherein the promoter is a non-specific promoter selected from the group of an hEF-1α promoter (SEQ ID NO: 5), a cytomegalovirus (CMV) promoter (SEQ ID NO: 6), a rous-sarcoma virus (RSV) promoter (SEQ ID NO: 7), a human ubiquitin C (hUBC) promoter (SEQ ID NO: 8), a simian vacuolating virus 40 (SV40) promoter (SEQ ID NO: 9), a phosphoglycerate kinase (PGK) promoter (SEQ ID NO: 10), a β-globin promoter, a NF-kB promoter, an EGR1 promoter, an elF4A1 promoter, an FerL promoter, a GAPDH promoter, a β-Kin promoter, a ROSA26 promoter, and a human surfactant protein C (hSP-C) promoter; and preferably wherein the promoter is an hEF-1 a promoter (SEQ ID NO: 5).

11. The method of claim 3, wherein the viral vector is administered directly into the spinal parenchyma of the subject, into the intrathecal space of the subject, into the spinal subpial space of the subject, or into a peripheral spastic muscle of the subject, or into one or more dermatomes of the subject.

12. (canceled)

13. A method of treating spasticity in a subject comprises administrating to the subject a therapeutically effective amount of a viral vector comprising a polynucleotide encoding GAD, thereby treating spasticity in the subject.

14. The method of claim 13, wherein the viral vector is a defective viral vector derived from HSV comprising an expression cassette comprising the polynucleotide encoding GAD is inserted in the LAT (Latency Associated Transcripts) locus of the defective viral vector.

15. (canceled)

16. (canceled)

17. The method of claim 14, wherein the expression cassette comprises a promoter.

18. The method of claim 17, wherein the promoter is an afferent neuron-specific promoter selected from promoters of genes coding for sensory neuroreceptors, preferably a promoter of the TRP gene family, more preferably the promoter of TRPV1 or TRPM8; or promoters of genes coding for sensory neuromodulators or sensory neurotransmitters, preferably promoter of substance P, PACAP, CGRP, ADVL, more preferably promoter of CGRP, ADVL.

19. The method of claim 17, wherein the promoter is a non-specific promoter selected from the group of an hEF-1 alpha promoter, a cytomegalovirus (CMV) promoter, a rous-sarcoma virus (RSV) promoter, a human ubiquitin C (hUBC) promoter, a simian vacuolating virus 40 (SV40) promoter, a phosphoglycerate kinase (PGK) promoter, a β-globin promoter, a NF-kB promoter, an EGR1 promoter, an elF4A1 promoter, an FerL promoter, a GAPDH promoter, a β-Kin promoter, a ROSA26 promoter, and a human surfactant protein C (hSP-C) promoter; and preferably wherein the promoter is an hEF-1 a promoter.

20. The method of claim 13, wherein the viral vector is administered directly into the spinal parenchyma of the subject, into the intrathecal space of the subject, into the spinal subpial space of the subject, or into a peripheral spastic muscle of the subject, or into one or more dermatomes of the subject.

21. (canceled)

22. A treatment regimen for treating a subject having spasticity or a condition associated with spasticity comprises administrating a viral vector comprising a polynucleotide encoding GAD, wherein GAD is expressed, thereby treating the spasticity or the condition associated with spasticity.

23. The treatment regimen of claim 22, wherein the viral vector is a defective viral vector derived from HSV comprising an expression cassette comprising the polynucleotide encoding GAD is inserted in the LAT (Latency Associated Transcripts) locus of the defective viral vector.

24. (canceled)

25. (canceled)

26. The treatment regimen of claim 23, wherein the expression cassette comprises a promoter.

27. The treatment regimen of claim 26, wherein the promoter is an afferent neuron-specific promoter selected from promoters of genes coding for sensory neuroreceptors, preferably a promoter of the TRP gene family, more preferably the promoter of TRPV1 or TRPM8; or promoters of genes coding for sensory neuromodulators or sensory neurotransmitters, preferably promoter of substance P, PACAP, CGRP, ADVL, more preferably promoter of CGRP, ADVL.

28. The treatment regimen of claim 26, wherein the promoter is a non-specific promoter selected from the group of an hEF-1α promoter, a cytomegalovirus (CMV) promoter, a rous-sarcoma virus (RSV) promoter, a human ubiquitin C (hUBC) promoter, a simian vacuolating virus 40 (SV40) promoter, a phosphoglycerate kinase (PGK) promoter, a β-globin promoter, a NF-kB promoter, an EGR1 promoter, an elF4A1 promoter, an FerL promoter, a GAPDH promoter, a β-Kin promoter, a ROSA26 promoter, and a human surfactant protein C (hSP-C) promoter; and preferably wherein the promoter is an hEF-1 a promoter.

29. The treatment regimen of claim 22, wherein the viral vector is administered directly into the spinal parenchyma of the subject, into the intrathecal space of the subject, into the spinal subpial space of the subject, or into a peripheral spastic muscle of the subject, or into one or more dermatomes of the subject.

30. (canceled)


SEQUENCE LISTING (10 sequences)

SEQ ID NO: 1 - DNA, 1785 bp, Homo sapiens
  source 1..1785: mol_type = genomic DNA
atggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcagggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatatctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaa

SEQ ID NO: 2 - protein, 594 aa, Homo sapiens
MASSTPSSSATSSNAGADPNTTNLRPTTYDTWCGVAHGCTRKLGLKICGFLQRTNSLEEKSRLVSAFRERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 3 - DNA, 2400 bp, Homo sapiens
  source 1..2400: mol_type = genomic DNA
agctcgcccgcagctcgcactcgcaggcgacctgctccagtctccaaagccgatggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctaggtagacaattaagttgtcacaaactgtgtgaatgtatttgtagtttgttccaaagtaaatctatttctatattgtggtgtcaaagtagagtttaaaaattaaacaaaaaagacattgctccttttaaaagtcctttcttaagtttagaatacctctctaagaattcgtgacaaaaggctatgttctaatcaataaggaaaagcttaaaattgttataaatacttcccttacttttaatatagtgtgcaaagcaaactttattttcacttcagactagtaggactgaatagtgccaaattgcccctgaatcataaaaggttctttggggtgcagtaaaaaggacaaagtaaatataaaatatatgttgacaataaaaactcttgcctttttcatagtattagaaaaaaatttctaatttacctatagcaacatttcaaatgtatttaaatacatataattttacaaaaggaaaatatatatattaaaaaagatatcctattttgtaacatatagatttttattttatataggttatacaaactgcgggggcggaatt

SEQ ID NO: 4 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 5 - DNA, 1179 bp, Homo sapiens
  source 1..1179: mol_type = genomic DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 6 - DNA, 508 bp, Cytomegalovirus
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - DNA, 522 bp, Rous sarcoma virus
  source 1..522: mol_type = other DNA
ggtgcacaccaatgtggtgaatggtcaaatggcgtttattgtatcgagctaggcacttaaatacaattatctctgcaatgcggtattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcag

SEQ ID NO: 8 - DNA, 1212 bp, Homo sapiens
  source 1..1212: mol_type = genomic DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 9 - DNA, 331 bp, Simian vacuolating virus 40
  source 1..331: mol_type = genomic DNA
gtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 10 - DNA, 500 bp, Homo sapiens
  source 1..500: mol_type = genomic DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg